(19) United States
(12) Patent Application Publication
(10) Pub. No.: US 2014/0348781 A1
(43) Pub. Date: Nov. 27, 2014

(54) CONJUGATES OF GM-CSF AND IL-9, COMPOSITIONS AND METHODS RELATED THERETO

(71) Applicants: Jacques Galipeau, Atlanta, GA (US); Pingxin Li, Atlanta, GA (US)

(72) Inventors: Jacques Galipeau, Atlanta, GA (US); Pingxin Li, Atlanta, GA (US)

(73) Assignees: CHILDREN'S HEALTHCARE OF ATLANTA, INC, Atlanta, GA (US); EMORY UNIVERSITY, Atlanta, GA (US)

(21) Appl. No.: 14/282,106

(22) Filed: May 20, 2014

Related U.S. Application Data
(60) Provisional application No. 61/826,252, filed on May 22, 2013.

(51) Int. Cl. A61K 38/20; A61K 39/00; A61K 38/19; C07K 14/54; C07K 14/535

(52) U.S. Cl. CPC A61K 38/206; C07K 14/5425; C07K 14/535; A61K 38/193; A61K 39/00; A61K 2039/55527; A61K 2039/55522
USPC 424/85.2; 530/351; 536/23.5; 435/320.1; 435/354

(57) ABSTRACT

In certain embodiments, this disclosure relates to conjugates comprising GM-CSF and IL-9 and uses related thereto, e.g., enhancing the adaptive immune system. Typically the GM-CSF and IL-9 are connected by a polymer linker, e.g., polypeptide. In certain embodiments, the disclosure relates to nucleic acids encoding these polypeptide conjugates, vectors comprising nucleic acid encoding polypeptide conjugates, and protein expression systems comprising these vectors such as infectious viral particles and host cells comprising such a nucleic acids.

### CROSS REFERENCE TO RELATED APPLICATIONS

This application claims priority to U.S. Provisional Application No. 61/826,252 filed May 22, 2013, hereby incorporated by reference in its entirety.

This invention was made with government support under Grant No. A1093881 awarded by the National Institutes of Health. The Government has certain rights in the invention.

### BACKGROUND

Although many viruses are cleared by the immune system, certain retroviruses, like HIV, evade the immune system, lie dormant, spread, and cause chronic infections. The World Health Organization estimates that AIDS has killed more than 25 million people since it was first recognized. In 2007, there were 2.7 million new HIV infections and 2 million HIV-related deaths. Anti-retroviral drugs are medications for the treatment of infection by retroviruses. When several antiviral agents are taken in combination with a retroviral drug, the approach is known as highly active antiretroviral therapy (HAART). Although HAART may improve symptoms associated with infection, there is currently no cure for HIV. HAART can also have serious side-effects. Regimens can be complicated, requiring patients to take several pills at various times during the day. If patients miss a dose, drug resistance can develop. Therefore, there remains a need for improved antiviral therapies. In particular, there remains a need for antiviral therapies with reduced toxicity and improved efficacy over existing treatments.

Analogous to virus, cancer is thought to occur as a result of an immune system that is not properly removing uncontrolled proliferating cancer cells. Stimulating the immune system to recognize and eliminate cancerous cells has become a promising strategy for therapeutic treatments. For example, Provenge™ is a FDA-approved autologous cellular immunotherapy treatment. Peripheral blood leukocytes of a subject are harvested via leukapheresis. These enriched monocytes are incubated prostatic acid phosphatase (PAP) conjugated to cytokine granulocyte macrophage colony stimulating factor (PAP-GM-CSF). GM-CSF is thought to direct the target antigen to receptors on DC precursors, which then present PAP on their cell surface in a context sufficient to activate T cells for the cells that express PAP. Activated, PAP presenting DCs are administered to the subject to elicit an immune response retarding cancer growth. This strategy requires isolation and expansion of cells of the subject, and typically treatment does not entirely clear the subject of cancer or tumors. Thus, there is a need to identify improved methods.

Williams and Galipeau report GM-CSF-based fusion cytokines as ligands for immune modulation. See J Immunol. 2011, 186 (10):5527-32. Deng & Galipeau report reprogramming of B cells into regulatory cells with engineered fusokines. See Infect Disord Drug Targets, 2012, 12 (3):248-54. See also Hsieh et al., Mol Ther., 2012, 20 (9):1767-77, Williams et al. Mol Ther, 2010, 18: 1293-1301, Rafei et al., Nat Med, 2009, 15: 1038-1045, Stagg et al., Cancer Res, 2004, 64: 8795-8799, Sonoda et al., Blood, 1994, 84 (12): 4099-4106, Williams and Park, Cancer, 1991, 67 (10 Suppl):2705-7, WO 2005/0053579, WO 2005/026820, WO 2008/0014612, and U.S. Pat. Nos. 5,108,910, 7,323,549 and 7,217,421.

References cited herein are not an admission of prior art.

### SUMMARY

In certain embodiments, this disclosure relates to conjugates comprising a polypeptide of GM-CSF and a polypeptide IL-9. Typically the GM-CSF and IL-9 are connected by a polymer linker, e.g., polypeptide. In certain embodiments, the disclosure relates to nucleic acids encoding these polypeptide conjugates, vectors comprising nucleic acid encoding polypeptide conjugates, and protein expression systems comprising these vectors such as infectious viral particles and host cells comprising such a nucleic acids.

In certain embodiments, the disclosure relates to a polypeptide of GM-CSF and a polypeptide IL-9 further conjugated to one or more of the group selected from an adjuvant, a cytokine, a co-stimulatory molecule, an antigen, a protein, saccharide, polysaccharide, and a glycoprotein.

In certain embodiments, the disclosure relates to pharmaceutical compositions comprising conjugates and vectors disclosed herein and a pharmaceutically acceptable excipient. In certain embodiments, the disclosure relates to vaccines comprising conjugates and vectors disclosed herein and an antigen and optionally an adjuvant. Typically, the antigen is live attenuated virus, killed virus, a virus-like particle, virosome, cancerous cell, lipid bilayer structure with a surface antigen, viral protein or glycoprotein, bacteria, or bacterial antigen, or tumor associated antigen.

In certain embodiments, the disclosure relates to methods of treating or preventing a viral, bacterial, or parasitic infection comprising administering an effective amount of a pharmaceutical composition comprising a conjugate or vector disclosed herein optionally in combination with an antigen or vaccine and optionally an adjuvant. In certain embodiments, the subject is at risk or, exhibiting symptoms of, or diagnosed with a viral infection, such as a chronic viral infection.

In certain embodiments, the disclosure relates to methods of treating or preventing a viral infection comprising administering an effective amount of a vaccine comprising a conjugate disclosed herein to a subject in need thereof.

In certain embodiments, the subject is diagnosed with influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubellavirus, lymphocytic choriomeningitis virus (LCMV), yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV).

In certain embodiments, the disclosure relates to administering a conjugate or vector disclosed herein in combination with another antiviral agent such as abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, and/or zidovudine.

In certain embodiments, the disclosure relates to methods of treating or preventing cancer comprising administering a pharmaceutical composition comprising a conjugate or vector disclosed herein to a subject in need thereof.

In certain embodiments, the disclosure relates to methods of treating or preventing cancer comprising administering autologous blood cells activated with a cancer antigen conjugated to GM-CSF in combination with a conjugate disclosed herein to a subject in need thereof.

In certain embodiments, the disclosure relates to methods of activating peripheral blood cells comprising mixing peripheral blood cells with a conjugate disclosed herein comprising a tumor associated antigen/cancer marker under conditions such that increase expression of CD54 occurs. In certain embodiments, the disclosure relates to product produced by mixing peripheral blood cells and with a conjugate disclosed herein under conditions such that increase expression of CD54 occurs. In certain embodiments, the disclosure relates to methods of treating or preventing cancer comprising administering an effective amount of a product made by mixing peripheral blood cells with a conjugate disclosed herein to subject from whom the peripheral blood cells were obtained.

In some embodiments, the disclosure relates to a method of treating or preventing cancer comprising by administering a pharmaceutical composition comprising conjugates or vector disclosed herein to a subject diagnosed with, exhibiting symptoms of, or at risk of cancer wherein the cancer is a hematological malignancy such as a leukemia or lymphoma, acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), Hodgkin's lymphomas, and non-Hodgkin's lymphomas such as Burkitt lymphoma, B-cell lymphoma and multiple myeloma. Other contemplated cancers include cervical, ovarian, colon, breast, gastric, lung, skin, ovarian, pancreatic, prostate, head, neck, and renal cancer.

Within any of the cancer management methods disclosed herein, the conjugate or vector may be administered in combination with an anti-cancer agent such as gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vincristine, vinblastine, vindesine, vinorelbine taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, bevacizumab, combretastatin, thalidomide, and/or lenalidomide or combinations thereof.

In certain embodiments, the disclosure relates to gene therapies comprising administering vectors comprising nucleic acid encoding conjugates disclosed herein to a subject in need thereof.

In certain embodiments, the disclosure contemplates incorporating conjugates disclosed herein into the surfaces of particles, e.g., cells, liposomes, micelles, vesicles, bilayer structures, virosomes, and virus-like particles. The conjugates may be linked to lipophilic moieties, e.g., fatty acids and GPI. In one example, the disclosure contemplates a GPI anchored conjugate comprising GPI, GM-CSF, IL-9, and optionally an antigen, adjuvant, or other polypeptide. It is contemplated that these particles may contain other surface polypeptides, antigens and co-stimulatory molecules such as B7-1, B7-2, ICAM-1, and/or IL-2. It is contemplated that these particles may be used in all the applications conjugates disclosed herein are mentioned.

Within certain embodiments, any of the conjugates disclosed herein may be further conjugated to an adjuvant, cytokine, co-stimulatory molecule, antigen, protein, or glycoprotein. In certain embodiments, the antigen is a viral protein or a cancer marker.

In certain embodiments, the cancer marker is selected from PAP (prostatic acid phosphatase), prostate-specific antigen (PSA), (PSMA) prostate-specific membrane antigen, early prostate cancer antigen-2 (EPCA-2), AKAP-4 (A kinase [PRKA] anchor protein 4), NGEP (new gene expressed in prostate), PSCA (prostate stem cell antigen), STEAP (six-transmembrane epithelial antigen of the prostate), MUC 1 (mucin 1), HER-2, BCL-2, MAGE antigens such as CT7, MAGE-A3 and MAGE-A4, ERK5, G-protein coupled estrogen receptor 1, CA15-3, CA19-9, CA 72-4, CA-125, carcinoembryonic antigen, CD20, CD31, CD34, PTPRC (CD45), CD99, CD117, melanoma-associated antigen (TA-90), peripheral myelin protein 22 (PMP22), epithelial membrane proteins (EMP-1, -2, and -3), HMB-45 antigen, MART-1 (Melan-A), S100A1, and S100B or fragments or mutated forms thereof.

In certain embodiments, the viral antigen is selected from an influenza virus hemagglutinin and neuraminidase; cytomegalovirus glycoprotein gB, p28, p38, p50, p52, p65, and p150; Borrelia p41; HIV nef, integrase, gag, protease, tat, env, p31, p17, p24, p31, p55, p66, gp32, gp36, gp39, gp41, gp120, and gp160; SIV p55; HBV core, surface antigen, and australian antigen; HCV core nucleocapsid, NS3, NS4, and NS5; Dengue env and NS1; EBV early antigen, p18, p23, gp125, nuclear antigen (EBNA)-1, EBNA-2, EBNA-3A, EBNA-3B, EBNA-3C, EBNA-leader protein (EBNA-LP), latent membrane proteins (LMP)-1, LMP-2A and LMP-2B; and herpes simplex virus gD and gG or fragments or mutated forms thereof.

In certain embodiments, the adjuvant or cytokine is selected from IL-2, IL-12, IL-15, IL-18, IL-21, IL-27, IL-31, IFN-alpha, flagellin, unmethylated, CpG oligonucleotide, lipopolysaccharides, lipid A, and heat stable antigen (HSA).

In certain embodiments, the disclosure relates to the production of a medicament comprising GM-CSF and IL-9 conjugates disclosed herein for uses disclosed herein.

### BRIEF DESCRIPTION OF THE FIGURES

FIG. 1 illustrates mouse GIFT9 protein expression and biochemical analyses. (A) GIFT9 amino acid sequence (SEQ ID NO: 2). The signal peptides of each part are underscored, IL9 signal peptide serves as the linker of the two parts. (B) Western blot of GIFT9 protein in the condition media from 293T cells retrovirally transduced to express GIFT9. Recombinant mouse GMCSF and IL9 were used as controls. (C) Western Blot of phospho-STAT1, STAT3, and STAT5 in JawsII cells after GIFT9 stimulation. Total STAT protein was used as a loading control. (D) Western Blot of phospho-STAT1, STAT3, and STAT5 in MC/9 cells after GIFT9 stimulation without or with GMCSF-Rα antibody blocking. Total STAT protein was used as a loading control.

FIG. 2 shows data indicating GIFT9 stimulation did not increase JAK kinase activity and receptor polarization to lipid rafts is not the major mechanism of GIFT9-induced hyperphosphorylation of STAT1. (A) JAK1, JAK2, and JAK3 phosphorylation levels remain same after GIFT9 stimulation. Total JAK protein was used as a loading control. (B) Inhibition of lipid rafts did not affect the hyperphosphorylation of STAT1 induced by GIFT9. Total STAT protein was used as a loading control.

FIG. 3 shows data indicating GMCSFR and IL9R co-localize after GIFT9 treatment. (A) GMCSFR βc was co-immunoprecipitated by α-common γc antibody after GIFT9 stimulation. (B) Common γc was co-immunoprecipitated by α-GMCSF-Rβ antibody after GIFT9 stimulation. (C) Representative confocal microscopy images of immunofluorescence staining showed the colocalization of GMCSF receptor and IL9 receptor after GIFT9 stimulation. Scale bar: 5 mm. (D) Percentages of color pixel colocalization after different cytokine treatment.

FIG. 4 shows data indicating JAK2 transphosphorylates STAT1 after GIFT9 treatment. (A) JAK2 inhibitor TG101348 treatment abolished hyperphosphorylation of STAT1 after GIFT9 stimulation. Total STAT protein was used as a loading control. (B) JAK3 inhibitor CP690550 treatment had a minor effect of STAT1 hyperphosphorylation induced by JAK2. Total STAT protein was used as a loading control. (C) Double inhibition of JAK1 and JAK2 by INCB018424 inhibited STAT5 phosphorylation after GMCSF/IL9 but not GIFT9 stimulation. Total STAT protein was used as a loading control. (D) Inhibition of all JAKs by INCB018424 and CP690550 depleted STATs phosphorylation. Total STAT protein was used as a loading control.

FIG. 5 shows data indicating GIFT9 stimulates the growth of BMMCs. (A) Flow cytometry analysis of BMMCs. (B) MTT assay of BMMCs after 5 days of culture with GMCSF/IL9 or GIFT9. (C) Model of GMCSF and IL9 receptor clustering and downstream signaling after GIFT9 stimulation.

### DETAILED DESCRIPTION

### GMCSF-IL-9 Fusokine (GIFT9)

In certain embodiments, provided herein is an engineered bifunctional cytokine borne of the fusion of Granulocyte Macrophage Colony Stimulating Factor (GMCSF) and Interleukin-9 (IL9) (hereafter GIFT9 fusokine). Human GIFT9 (hGIFT9) has the following amino acid sequence (SEQ ID NO: 1) (288aa)

MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQEMLLAMV
LTSALLLCSVAGQGCPTLAGILDINFLINKMQEDPASKCHCSANVTSCLC
LGIPSDNCTRPCFSERLSQMTNTTMQTRYPLIFSRVKKSVEVLKNNKCPY
FSCEQPCNQTTAGNALTFLKSLLEIFQKEKMRGMRGKI.

The hGIFT9 DNA sequence (867 bp) is SEQ ID NO:3

ATGTGGCTGCAGAGCCTGCTGCTCTTGGGCACTGTGGCCTGCAGCATCTC
TGCACCCGCCCGCTCGCCCAGCCCCAGCACGCAGCCCTGGGAGCATGTGA
ATGCCATCCAGGAGGCCCGGCGTCTCCTGAACCTGAGTAGAGACACTGCT
GCTGAGATGAATGAAACAGTAGAAGTCATCTCAGAAATGTTTGACCTCCA
GGAGCCGACCTGCCTACAGACCCGCCTGGAGCTGTACAAGCAGGGCCTGC
GGGGCAGCCTCACCAAGCTCAAGGGCCCCTTGACCATGATGGCCAGCCAC
TACAAGCAGCACTGCCCTCCAACCCCGGAAACTTCCTGTGCAACCCAGAT
TATCACCTTTGAAAGTTTCAAAGAGAACCTGAAGGACTTTCTGCTTGTCA
TCCCCTTTGACTGCTGGGAGCCAGTCCAGGAGATGCTTCTGGCCATGGTC
CTTACCTCTGCCCTGCTCCTGTGCTCCGTGGCAGGCCAGGGGTGTCCAAC
CTTGGCGGGGATCCTGGACATCAACTTCCTCATCAACAAGATGCAGGAAG
ATCCAGCTTCCAAGTGCCACTGCAGTGCTAATGTGACCAGTTGTCTCTGT
TTGGGCATTCCCTCTGACAACTGCACCAGACCATGCTTCAGTGAGAGACT
GTCTCAGATGACCAATACCACCATGCAAACAAGATACCCACTGATTTTCA
GTCGGGTGAAAAAATCAGTTGAAGTACTAAAGAACAACAAGTGTCCATAT
TTTTCCTGTGAACAGCCATGCAACCAAACCACGGCAGGCAACGCGCTGAC
ATTTCTGAAGAGTCTTCTGGAAATTTTCCAGAAAGAAAAGATGAGAGGGA
TGAGAGGCAAGATATGA.

Experiments herein indicated the GIFT9 chaperones co-clustering of the functionally unrelated GMCSF receptor (GMCSFR) and IL9 receptor (IL9R) on cell surface of target cells. GIFT9 treatment of MC/9 cells leads to transhyperphosphorylation of IL9R-associated STAT1 by GMCSFR-associated JAK2. IL9R-associated JAK1 and JAK3 augment phosphorylation of GMCSFR-linked STAT5. The functional relevance of these synergistic JAK/STAT transphosphorylation events translates to an increased mitogenic response by GMCSFR/IL9R-expressing primary marrow mast cells.

The GMCSF receptor (GMCSFR) contains two subunits: GMCSFRα chain and the common β chain (βc) that is shared with IL3 and IL5. IL 9 receptor (IL9R) is constituted by IL9Rα chain and the common γ chain that is shared with other γc family interleukins including IL2, IL4, IL7, IL15, and IL21. Both GMCSFR and γc interleukin receptors signal through JAK/STAT pathways. They bind to the intracellular domains of receptors in an unphosphorylated form, and are rapidly self-phosphorylated and activated upon the extracellular binding of ligands. The active forms of JAKs further phosphorylate specific tyrosine/serine residues on the receptors, leading the SH2-dependent recruitment of STATs, which are distributed in cellular cytoplasm as inactive homodimers. After receptor binding, STATs are activated through phosphorylation by JAKs. Activated STATs dissociate from the receptors and translocate to cell nucleus to start transcription. Each JAK associates with defined receptors and activate specific STATs as their substrates. For instance, GMCSFR βc associates with JAK2, and utilize STAT5 as its substrate. Unlike other JAKs that have broad expression patterns, the expression of JAK3 is typically restricted to leukocytes, and associates with interleukin γc receptors. The association of IL9 with its receptor activates the phosphorylation of JAK1 and JAK3, which leads to the activation of STAT1, STAT3, and STAT5. Experiments with the fusion of GMCSF and IL9 (GIFT9) indicate that GIFT9 is able to induce the functional clustering of GMCSF and IL9 receptors and alter their respective downstream STAT phosphorylation signals through reciprocal JAK/STAT transactivation.

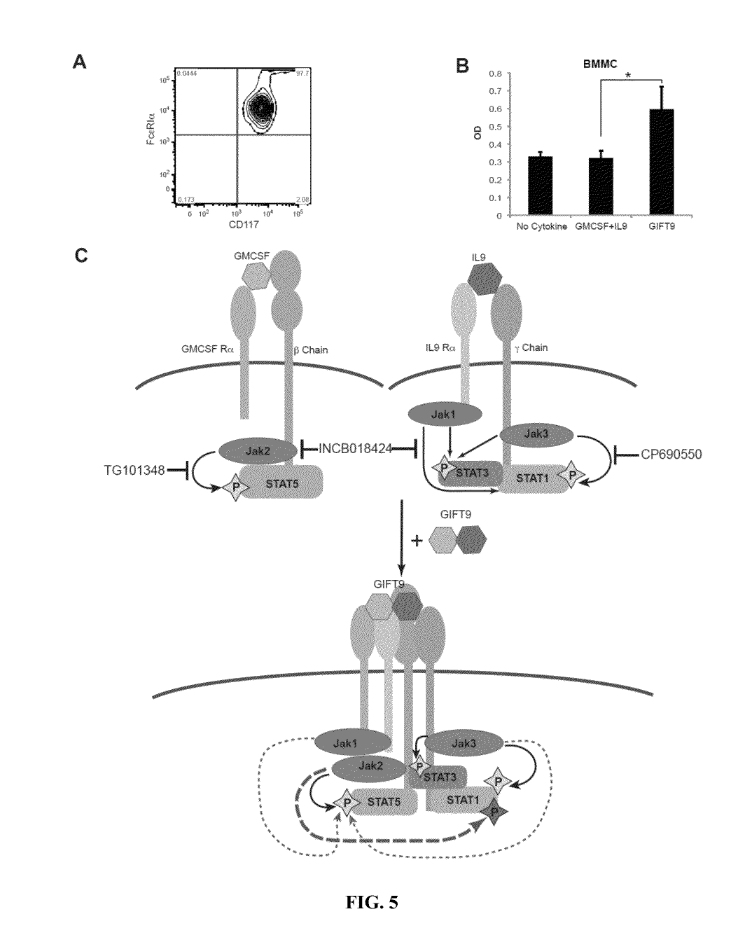

Although it is not intended that certain embodiments of the disclosure be limited by any particular mechanism, it is believed that the gain-of-function induced by GIFT9 treatment is due to the physical clustering of GMCSFR and IL9R with GIFT9 behaving as an extracellular activator/chaperone. Experiments indicate that GIFT9 leads to hyperphosphorylation of STAT1, with acquisition of redundant yet homeostatic STAT3 and STAT5 phosphorylation levels. The GIFT9-mediated hyperphosphorylation of STAT1 is neither due to the hyperactivation of JAKs, nor the polarization of GMCSFR and IL9R to membrane lipid rafts. Rather, GMCSFR/IL9R clustering enables GMCSFR-associated JAK2 to transphosphorylate IL9R-associated STAT1. Interestingly, STAT5 phosphorylation levels remained high after GIFT9 treatment when JAK2 kinase function is inhibited. Further experiments demonstrated that receptor clustering deployed IL9R-associated JAK1 and JAK3 kinase activities targeting GMCSFR-associated STAT5 (FIG. 5C).

Under physiological circumstances, unrelated cytokine receptors are randomly distributed on cell membrane surface. When ligand bound, individual receptors can migrate to lipid rafts where they may interact with each other. This may represent a mechanism by which a cell can integrate synergistically the signals mediated by its cytokine milieu. In an effort to translate this biological insight in to a medically useful therapeutic strategy, a synthetic bi-domain cytokine was tested for its ability to act as a chaperone and mediate receptor clustering and synergistic gain-of-function properties. The development of fusion cytokine proteins is potentially of importance since it provides a strategy to bring together receptors that are usually not in the same signaling complex, so as to trigger downstream events that cannot be achieved by other methods. As demonstrated herein by GIFT9, the design of fusion proteins can be extended cytokine coupling based on their targeting receptor expression pattern of a cell in order to elicit target specific gain-of-function pharmaceutical effects.

Data collected suggests use in the treatment of human disorders where an enhanced of immune response is desirable, in particular chronic infectious ailments and cancer. Moreover, GIFT9 or GIFT9-enhanced T cell precursors may be use of for the treatment of a non-exclusive listing of human immune deficient ailments such as congenital or HIV-mediated acquired immunodeficiency, after chemotherapy or after-hematopoietic stem cell transplant (HSCT) and for the ageing.

In certain embodiments, the present disclosure encompasses fusion proteins involving full-length pre-processed forms, as well as mature processed forms, fragments thereof and variants of each or both of the GM-CSF and IL-9 entities, including allelic as well as non-naturally occurring variants. In addition to naturally-occurring allelic variants of the GM-CSF and IL-9 entities that may exist in the population, the skilled artisan will further appreciate that changes (i.e. one or more deletions, additions and/or substitutions of one or more amino acid) can be introduced by mutation using classic or recombinant techniques to effect random or targeted mutagenesis. A suitable variant in use in the present disclosure typically has an amino acid sequence having a high degree of homology with the amino acid sequence of the corresponding native cytokine. In one embodiment, the amino acid sequence of the variant cytokine in use in the fusion protein of the disclosure is at least 70%, at least about 75%, at least about 80%, at least about 90%, typically at least about 95%, more typically at least about 97% and even more typically at least about 99% identical to the corresponding native sequence. In certain embodiments such native sequence is of human GM-CSF and/or human IL-9.

Percent identities between amino acid or nucleic acid sequences can be determined using standard methods known to those of skill in the art. For instance for determining the percentage of homology between two amino acid sequences, the sequences are aligned for optimal comparison purposes. The amino acid residues at corresponding amino acid positions are then compared. Gaps can be introduced in one or both amino acid sequence(s) for optimal alignment and non-homologous sequences can be disregarded for comparison purposes. When a position in the first sequence is occupied by the same amino acid residue as the corresponding position in the second sequence, then the sequences are identical at that position. The percent identity between the two sequences is a function of the number of identical positions shared by the sequences, taking into account the number of gaps which need to be introduced for optimal alignment and the length of each gap. The comparison of sequences and determination of percent identity and similarity between two sequences can be accomplished using a mathematical algorithm (e.g. Computational Molecular Biology, 1988, Ed Lesk A M, Oxford University Press, New York; Biocomputing: Informatics and Genome Projects, 1993, Ed Smith D. W., Academic Press, New York; Computer Analysis of Sequence Data, 1994, Eds Griffin A. M. and Griffin H. G., Human Press, New Jersey; Sequence Analysis Primer, 1991, Eds Griskov M. and Devereux J., Stockton Press, New York). Moreover, various computer programs are available to determine percentage identities between amino acid sequences and between nucleic acid sequences, such as GCG™ program (available from Genetics Computer Group, Madison, Wis.), DNAsis™ program (available from Hitachi Software, San Bruno, Calif.) or the MacVector™ program (available from the Eastman Kodak Company, New Haven, Conn.).

Suitable variants of GM-CSF and IL-9 entities for use in the present disclosure are biologically active and retain at least one of the activities described herein in connection with the corresponding polypeptide. Typically, the therapeutic effect (e.g. adjuvant effect) is preserved, although a given function of the polypeptide(s) may be positively or negatively affected to some degree, e.g. with variants exhibiting reduced cytotoxicity or enhanced biological activity. Amino acids that are essential for a given function can be identified by methods known in the art, such as by site-directed mutagenesis. Amino acids that are critical for binding a partner/substrate (e.g. a receptor) can also be determined by structural analysis such as crystallization, nuclear magnetic resonance and/or photoaffinity labeling. The resulting variant can be tested for biological activity in assays such as those described above.

For example, in one class of functional variants, one or more amino acid residues are conservatively substituted. A “conservative amino acid substitution” is one in which the amino acid residue in the native polypeptide is replaced with an amino acid residue having a similar side chain. Families of amino acid residues having similar side chains have been defined in the art. Typically, substitutions are regarded as conservative when the replacement, one for another, is among the aliphatic amino acids Ala, Val, Leu, and Ile; the hydroxyl residues Ser and Thr; the acidic residues Asp and Glu; the amide residues Asn and Gln; the basic residues Lys and Arg; or the aromatic residues Phe and Tyr. Alternatively, in another embodiment, mutations can be introduced randomly along all or part of a cytokine coding sequence, such as by saturation mutagenesis, and the resultant mutant can be screened for its biological activity as described herein to identify mutants that retain at least therapeutic activity.

Although the GM-CSF and IL-9 entities can be directly fused in the fusion protein of the disclosure, it is however typical to use a linker peptide for joining GM-CSF and IL-9. The purpose of the linker is to allow the correct formation, folding and/or functioning of each of the GM-CSF and IL-9 entities. It should be sufficiently flexible and sufficiently long to achieve that purpose. Typically, the coding sequence of the linker may be chosen such that it encourages translational pausing and therefore independent folding of the GM-CSF and IL-9 entities. A person skilled in the art will be able to design suitable linkers in accordance with the disclosure. The present disclosure is, however, not limited by the form, size or number of linker sequences employed. Multiple copies of the linker sequence of choice may be inserted between GM-CSF and IL-9. The only requirement for the linker sequence is that it functionally does not adversely interfere with the folding and/or functioning of the individual entities of the fusion protein. For example, a suitable linker is 5 to 50 amino acid long and may comprise amino acids such as glycine, serine, threonine, asparagine, alanine and proline (see for example Wiederrecht et al., 1988, Cell 54, 841; Dekker et al., 1993, Nature 362, 852; Sturm et al., 1988, Genes and Dev. 2, 1582; Aumailly et al., 1990 FEBS Lett. 262, 82). Repeats comprising serine and glycine residues are typical in the context of the disclosure. Specific examples of suitable linkers consists of two or three or more (e.g. up to eight) copies of the sequence Gly-Gly-Gly-Gly-Ser (GGGGS). It will be evident that the disclosure is not limited to the use of these particular linkers.

The disclosure further includes fusion proteins which comprise, or alternatively consist essentially of, or alternatively consist of an amino acid sequence which is at least 70%, 75%, 80%, 90%, 95%, 97%, 99% homologous or even better 100% homologous (identical) to all or part of any of the amino acid sequences recited in SEQ ID NO: 1-2.

In the context of the present disclosure, a protein “consists of” an amino acid sequence when the protein does not contain any amino acids but the recited amino acid sequence. A protein “consists essentially of” an amino acid sequence when such an amino acid sequence is present together with only a few additional amino acid residues, typically from about 1 to about 50 or so additional residues. A protein “comprises” an amino acid sequence when the amino acid sequence is at least part of the final (i.e. mature) amino acid sequence of the protein. Such a protein can have a few up to several hundred additional amino acids residues. Such additional amino acid residues can be naturally associated with each or both entities contained in the fusion or heterologous amino acid/peptide sequences (heterologous with respect to the respective entities). Such additional amino acid residues may play a role in processing of the fusion protein from a precursor to a mature form, may facilitate protein trafficking, prolong or shorten protein half-life or facilitate manipulation of the fusion protein for assay or production, among other things. Typically, the fusion proteins of the disclosure comprise a signal peptide at the NH2-terminus in order to promote secretion in the host cell or organism. For example, the endogenous signal peptide (i.e. naturally present in the cytokine present at the NH2 terminus of said fusion) can be used or alternatively a suitable heterologous (with respect to the cytokine in question) signal peptide sequence can be added to the cytokine entity present at the NH2 terminus of the fusion or inserted in replacement of the endogenous one.

In the context of the disclosure, the fusion proteins of the disclosure can comprise cytokine entities of any origin, i.e. any human or animal source (including canine, avian, bovine, murine, ovine, feline, porcine, etc). Although “chimeric” fusion proteins are also encompassed by the disclosure (e.g. one cytokine entity of human origin and the other of an animal source), it is typical that each entity be of the same origin (e.g. both from humans).

The fusion proteins of the present disclosure can be produced by standard techniques. Polypeptide and DNA sequences for each of the cytokines involved in the fusion protein of the present disclosure are published in the art, as are methods for obtaining expression thereof through recombinant or chemical synthetic techniques. In another embodiment, a fusion-encoding DNA sequence can be synthesized by conventional techniques including automated DNA synthesizers. Then, the DNA sequence encoding the fusion protein may be constructed in a vector and operably linked to a regulatory region capable of controlling expression of the fusion protein in a host cell or organism. Techniques for cloning DNA sequences for instance in viral vectors or plasmids are known to those of skill in the art (Sambrook et al, 2001, “Molecular Cloning. A Laboratory Manual”, Laboratory Press, Cold Spring Harbor N.Y.). The fusion protein of the disclosure can be purified from cells that have been transformed to express it.

The present disclosure also provides a nucleic acid molecule encoding the fusion protein of the disclosure. Within the context of the present disclosure, the term “nucleic acid” and “polynucleotide” are used interchangeably and define a polymer of nucleotides of any length, either deoxyribonucleotide (DNA) molecules (e.g., cDNA or genomic DNA) and ribonucleotide (RNA) molecules (e.g., mRNA) and analogs of the DNA or RNA generated using nucleotide analogs (see U.S. Pat. No. 5,525,711 and U.S. Pat. No. 4,711,955 as examples of nucleotide analogs). If present, modifications to the nucleotide structure may be imparted before or after assembly of the polymer. The sequence of nucleotides may also be interrupted by non-nucleotide elements. The nucleic acid molecule may be further modified after polymerization, such as by conjugation with a labeling component. The nucleic acid, especially DNA, can be double-stranded or single-stranded, but typically is double-stranded DNA. Single-stranded nucleic acids can be the coding strand (sense strand) or the non-coding strand (anti-sense strand).

The nucleic acid molecules of the disclosure include, but are not limited to, the sequence encoding the fusion protein alone, but may comprise additional non-coding sequences, for example introns and non-coding 5′ and 3′ sequences that play a role in transcription, mRNA processing (including splicing and polyadenylation signals), ribosome binding and mRNA stability. For example, the nucleic acid molecule of the disclosure can contain less than about 5 kb, 4 kb, 3 kb, 2 kb, 1 kb, 0.5 kb or 0.1 kb of nucleotide sequences which naturally flank (i.e. sequences located at the 5′ and 3′ ends) or are present within the genomic DNA encoding GM-CSF and IL-9 entities.

According to a typical embodiment, the present disclosure provides nucleic acid molecules which comprise, or alternatively consist essentially of, or alternatively consist of a nucleotide sequence encoding all or part of an amino acid sequence encoding a fusion protein which is at least about 70%, at least about 75%, at least about 80%, at least about 90%, at least about 95%, typically at least about 97%, more typically at least about 99% homologous or even more typically 100% homologous to any of the amino acid sequences shown in SEQ ID NO: 1-2.

In another embodiment, a nucleic acid molecule of the disclosure comprises a nucleic acid molecule which is a complement of all or part of a nucleotide sequence encoding the fusion protein shown in any of SEQ ID NO: 1-2. A nucleic acid molecule which is complementary to the nucleotide sequence of the present disclosure is one which is sufficiently complementary such that it can hybridize to the fusion-encoding nucleotide sequence under stringent conditions, thereby forming a stable duplex. Such stringent conditions are known to those skilled in the art. A typical, non-limiting example of stringent hybridization conditions are hybridization in 6 times sodium chloride/sodium citrate (SSC) at about 45 C, followed by one or more washes in 0.2 times SSC, 0.1% SDS at 50-65 C. In one embodiment, the disclosure pertains to antisense nucleic acid to the nucleic acid molecules of the disclosure. The antisense nucleic acid can be complementary to an entire coding strand, or to only a portion thereof.

In still another embodiment, the disclosure encompasses variants of the above-described nucleic acid molecules of the disclosure e.g., that encode variants of the fusion proteins that are described above. The variation(s) encompassed by the present disclosure can be created by introducing one or more nucleotide substitutions, additions and/or deletions into the nucleotide sequence by standard techniques, such as site-directed mutagenesis and PCR-mediated mutagenesis. Following mutagenesis, the variant nucleic acid molecule can be expressed recombinantly as described herein and the activity of the resulting protein can be determined using, for example, assays described herein. Alternatively, the nucleic acid molecule of the disclosure can be altered to provide preferential codon usage for a specific host cell (for example E. coli; Wada et al., 1992, Nucleic Acids Res. 20, 2111-2118). The disclosure further encompasses nucleic acid molecules that differ due to the degeneracy of the genetic code and thus encode for example the same fusion protein as any of those shown in SEQ ID NO: 1-2.

Another embodiment of the disclosure pertains to fragments of the nucleic acid molecule of the disclosure, e.g. restriction endonuclease and PCR-generated fragments. Such fragments can be used as probes, primers or fragments encoding an immunogenic portion of the fusion protein.

The nucleic acid molecules of the present disclosure can be generated using the sequence information provided herein. The nucleic acid encoding each of the GM-CSF and IL-9 entities can be cloned or amplified using cDNA or, alternatively, genomic DNA, as a template and appropriate probes or oligonucleotide primers according to standard molecular biology techniques (e.g., as described in Sambrook, et al. “Molecular Cloning: A Laboratory Manual”, Cold Spring Harbor Laboratory Press, Cold Spring Harbor, N.Y., 2001) or standard PCR amplification techniques based on sequence data accessible in the art (such as those provided above in connection with the fusion proteins of the disclosure or those provided in the Examples part). Fusing of the GM-CSF sequence to the IL-9 sequence may be accomplished as described in the Experimental below or by conventional techniques. For example, the GM-CSF and IL-9 encoding sequences can be ligated together in-frame either directly or through a sequence encoding a peptide linker. The GM-CSF-encoding sequence can also be inserted directly into a vector which contains the IL-9-encoding sequence, or vice versa. Alternatively, PCR amplification of the GM-CSF and IL-9-encoding sequences can be carried out using primers which give rise to complementary overhangs which can subsequently be annealed and re-amplified to generate a fusion gene sequence.

### GIFT9 Induces Heterologous Receptor Clustering and STAT1 Hyperactivation Through JAK2 Promiscuity

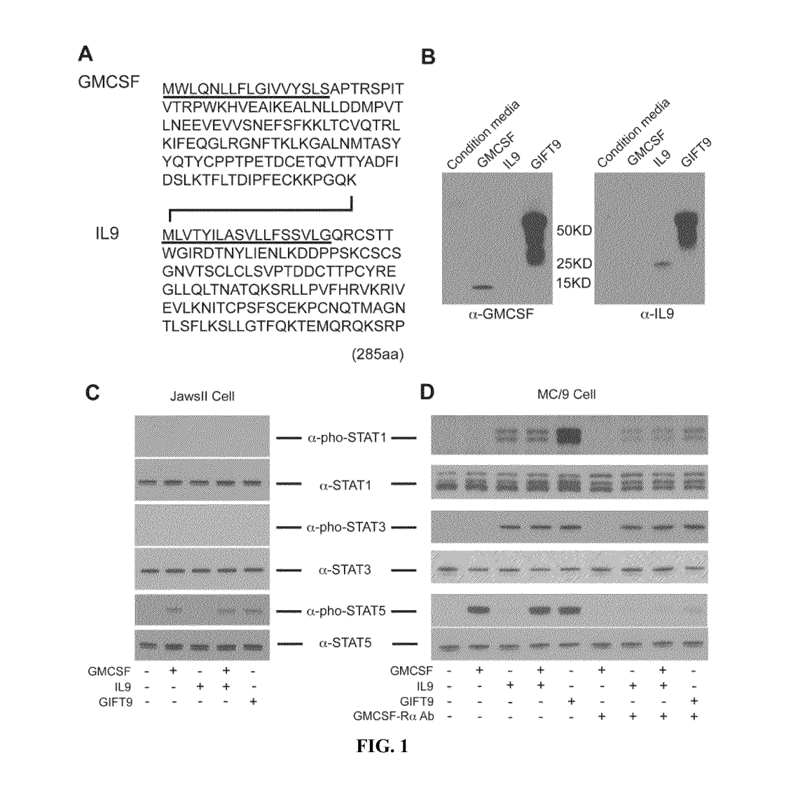

The GIFT9 fusion protein was created by aligning the cDNA encoding mouse GMCSF in frame with the 5′ end of the cDNA encoding mouse IL9. The stop codon of the mouse GMCSF cDNA was deleted, generating a cDNA encoding for a single 285 amino acid chain (FIG. 1A). Western blots were performed using the conditioned media of 293T cells retrovirally transduced to express GIFT9. Both anti-GMCSF and anti-IL9 antibodies recognized the same secreted protein at a molecular weight (MW) of ˜50 kD (FIG. 1B). The molecular weight spread of GIFT9 protein observed on SDS gel electrophoresis are likely due to the glycosylation of the fusion protein, as it has been previously reported that both GMCSF and IL9 can be glycosylated at multiple sites.

To study the cellular biochemistry of GIFT9 on GMCSFR signaling, GMCSF responsive JawsII cells were stimulated by GIFT9 for 15 minutes. STAT5 phosphorylation levels were similar to that arising from either GMCSF alone or GMCSF and IL9 combination (FIG. 1C). STAT1 and STAT3 phosphorylation levels remained undetectable as GMCSF signals through JAK2-STAT5 pathway. These data demonstrate that the GMCSF domain of GIFT9 is fully bioactive and deploys the canonical signaling properties of native GMCSF when bound to GMCSFR and that JawsII cells bereft of IL9R do not initiate γc interleukin driven STAT1/3 activation.

Upon treating GMCSFR⁺ IL9R⁺ MC/9 mast cells with GIFT9 for 15 minutes, a robust hyperphosphorylation of STAT1 was observed compared to controls, while phosphorylation of STAT3 and STAT5 remained similar (FIG. 1D). Since MC/9 cells express both GMCSFR and IL9R, the GMCSF-mediated signaling pathway was blocked by anti-GMCSFRα antibody to interrogate whether the hyperphosphorylation of STAT1 is solely due to the IL9/IL9R interaction. After blocking GMCSFRα, the hyperphosphorylation of STAT1 after GIFT9 stimulation was significantly decreased to a comparable level as IL9 stimulation alone (FIG. 1D). These data suggest that the gain-of-function GIFT9-mediated hyperphosphorylation of STAT1 depends upon GMCSFR cooperativity with IL9R signaling pathways. Furthermore, the blocking of GMCSFR signaling pathway robustly suppressed STAT5 phosphorylation, reinforcing the role of GMCSFR in STAT5 activation in these cells.

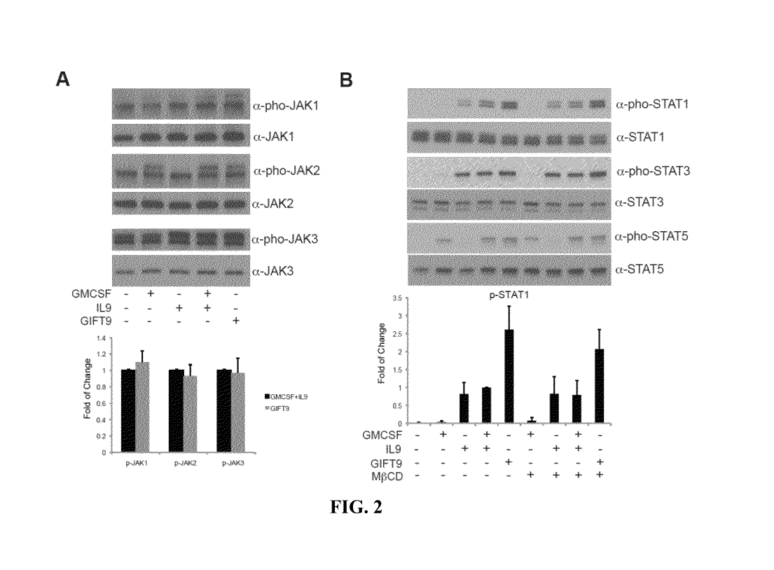

Since STAT1 phosphorylation is dependent upon JAK1 kinase activity, it is believed that the hyperphosphorylation of STAT1 observed with GIFT9 stimulation of MC/9 cells was due to the hyperactivation of IL9R-associated JAK1. Western blot performed to examine the level of JAK1 phosphorylation after GIFT9 stimulation suggests that GIFT9 induced comparable levels of phosphorylated JAK1 as the combination treatment of GMCSF and IL9 (FIG. 2A). Similar results were also obtained for JAK2 and JAK3 phosphorylation (FIG. 2A), demonstrating that the hyperactivation of STAT1 was not due to hyperactivation of GMCSFR and IL9R associated JAK kinases.

Lipid rafts play an important role in supporting transphosphorylation between distinct cytokine receptors through receptor clustering. Therefore, it is thought that lipid rafts may be important sites for recruiting GMCSFR and IL9R to induce hyperphosphorylation of STAT1 after GIFT9 treatment. To test this idea, MC/9 cells were pre-incubated with the lipid raft disruptor MβCD, followed by GIFT9 stimulation. Western blot analysis revealed that STAT1 phosphorylation after IL9 alone or GMCSF and IL9 double treatment remained similar after the disruption of lipid rafts (FIG. 2B), suggesting that an alternate mechanism other than lipid raft polarization is involved in inducing GIFT9-mediated STAT1 hyperactivation.

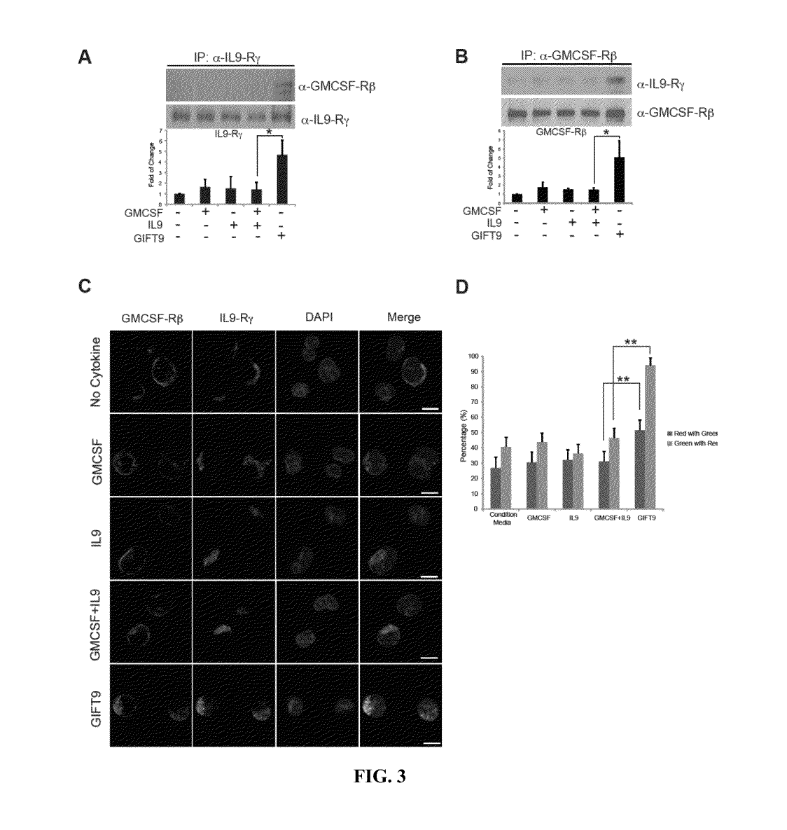

It is thought that GIFT9 bioactivity may arise by its ability to chaperone the clustering of GMCSFR and IL9R independently of lipid raft polarization. To test this possibility, a co-immunoprecipitation approach was utilized to test whether the IL9R γc and GMCSFR βc were physically associated when cells are treated with GIFT9. Following treatment of target MC/9 cells with GIFT9, the interleukin γc was immunoprecipitated and its associated proteins were probed for GMCSFR βc by Western blot. The co-immunoprecipitation assay showed that a significantly increased amount of GMCSFR βc was pulled down with IL9 γc after GIFT9 treatment (FIG. 3A), while only background level of GMCSFR βc was pulled down by IL9R γc after single or double cytokine treatment. These data suggest that GIFT9 chaperones the clustering of GMCSFR and IL9R into a stable complex. The reciprocal co-immunoprecipitation experiment was also performed, and similar results were observed (FIG. 3B).

To provide further evidence to support the hypothesis of GIFT9-mediated clustering of GMCSFR and IL9R, MC/9 cells were double stained with GMCSFR and IL9R antibodies after cytokine treatments and immunofluorescent images were visualized by confocal microscopy. After single or double GMCSF and IL9 treatment, GMCSFR and IL9R were not meaningfully co-localized, whilst receptor co-localization was evident after GIFT9 treatment (FIG. 3C). Analyses of individual cells in each treatment group demonstrated that the percentage of color pixel co-localization are significantly higher, approaching unity, in the GIFT9-treated group comparing with controls (FIG. 3D).

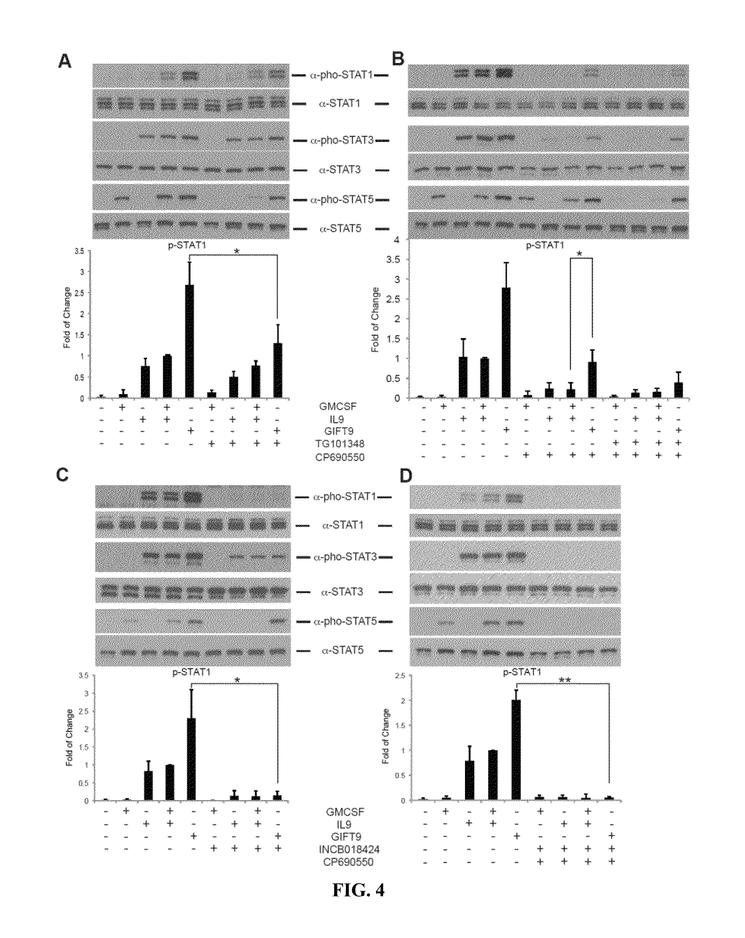

The physical evidence of GIFT9-mediated co-localization of GMCSFR and IL9R provides a testable explanation for the observed hyperphosphorylation of IL9R-associated STAT1 in MC/9 cells. STAT1 hyperactivation may be due to transphophorylation by promiscuous JAK2 activity that is normally associated with GMCSFR. Indeed, STAT1 is known to be a JAK2 target in other circumstances. To test this idea, MC/9 cells were pre-treated with the JAK2 inhibitor TG101348 for 5 hours and STAT activation levels were examined. After JAK2 inhibition, GIFT9 induced STAT1 phosphorylation decreased to a comparable level as GMCSF and IL9 double treatment, while STAT3 phosphorylation remained unchanged (FIG. 4A). These data support a theory that GMCSFR associated JAK2 transphosphorylates IL9R-associated STAT1. Different from STAT1 phosphorylation, STAT3 phosphorylation level remains similar after GIFT9 treatment although JAK2 has been reported to be able to phophorylate STAT3. This could be due to that STAT3 phosphorylation already reaches its maximum level driven by JAK1/3, as STAT3 phosphorylation remained unchanged when JAK2 was inhibited but decreased when JAK3 were inhibited after GIFT9 treatment (FIG. 4A-B). Interestingly, STAT5 phosphorylation levels were nearly abolished in single and double cytokine treated samples but not in GIFT9 treated cells (FIG. 4A), suggesting that other JAKs may deploy STAT5 tropism when dominant JAK2 is blocked. To test whether other JAKs are involved in transphosphorylation of STAT5, JAK3 function was first inhibited by pre-incubating MC/9 cells with the JAK3 inhibitor CP690550. Following JAK3 inhibition, STAT1 phosphorylation decreased after GIFT9 and abolished STAT1 activation was observed in GMCSF and GMCSF⁺ IL9 double treatment groups (FIG. 4B). STAT3 phosphorylation decreased as well, which is consistent with previous studies as JAK3-STAT3 signals through the IL9R γc. These data suggested that JAK3 phosphorylates STAT1 in a canonical response to IL9. However, GIFT9 provides a redundant STAT1 activation pathway afforded by JAK2. To validate the hypothesis that GIFT9 leads to STAT1 hyperphosphorylation through the activities of both JAK2 and JAK3, JAK2 and JAK3 activities were inhibited jointly by the use of both TG101348 and CP690550 simultaneously. Dual JAK2/JAK3 inhibition abolished STAT1/3/5 activation in MC/9 cells following treatment with GMCSF IL9, yet meaningful activation of all STATs was still apparent following GIFT9 treatment (FIG. 4B). These data suggest a possible role for JAK1 kinase activity deployed by GIFT9 activation. To interrogate JAK1 activity, the JAK1/2 inhibitor INCB018424 was used to treat the MC/9 cells before cytokine stimulation. Western blot showed that when both JAK1 and JAK2 were inhibited, STAT1 phosphorylation reduced to basal levels and STAT3 phosphorylation decreased modestly, likely reflecting residual JAK3 activity (FIG. 4C). Since STAT1/3/5 phosphorylation persists after GIFT9 treatment despite JAK2 and JAK3 co-inhibition, all three JAKs were inhibited by using INCB018424 and CP690550 simultaneously to determine whether other non-canonical kinases contributed to STAT phosphorylation in response to GIFT9. Western blot analysis reveals that GIFT9-mediated STAT1, STAT3 and STAT5 phosphorylation were completely abolished when JAK1/2/3 were inhibited contemporaneously (FIG. 4D), confirming that virtually all the GIFT9-mediated gain-of-function STAT phosphorylation events are mediated solely through GMCSFR and IL9R associated JAK1/2/3.

CD 117⁺ FcεRIα⁺ mast cells are IL9-responsive. To validate whether GIFT9-mediated receptor clustering leads to meaningful gain of function in non-transformed normal myeloid cells, whether bone marrow derived mast cells, deployed an alternate functionality following exposure to GIFT9 in vitro was tested. Unfractionated bone marrow cells from C57BL/6J mice were harvested and culture expanded primary mast cells with IL-3 and Stem Cell Factor. Flow cytometry analysis showed that bone marrow mast cell cultures had over 97% mast cells at the time of use (FIG. 5A). Mast cells were subsequently treated with GIFT9 and proliferation rates were compared to controls. It was found that mast cells treated with GIFT9 significantly increased their proliferative rate by 100% (FIG. 5B).

### Pharmaceutical Compositions

As used herein the language “pharmaceutically acceptable excipient” is intended to include any and all carriers, solvents, diluents, excipients, adjuvants, dispersion media, coatings, antibacterial and antifungal agents, and absorption delaying agents, and the like, compatible with pharmaceutical administration.

Suitably, the pharmaceutical composition of the disclosure comprises a carrier and/or diluent appropriate for its delivering by injection to a human or animal organism. Such carrier and/or diluent is non-toxic at the dosage and concentration employed. It is selected from those usually employed to formulate compositions for parental administration in either unit dosage or multi-dose form or for direct infusion by continuous or periodic infusion. It is typically isotonic, hypotonic or weakly hypertonic and has a relatively low ionic strength, such as provided by sugars, polyalcohols and isotonic saline solutions. Representative examples include sterile water, physiological saline (e.g. sodium chloride), bacteriostatic water, Ringer's solution, glucose or saccharose solutions, Hank's solution, and other aqueous physiologically balanced salt solutions (see for example the most current edition of Remington: The Science and Practice of Pharmacy, A. Gennaro, Lippincott, Williams & Wilkins). The pH of the composition of the disclosure is suitably adjusted and buffered in order to be appropriate for use in humans or animals, typically at a physiological or slightly basic pH (between about pH 8 to about pH 9, with a special preference for pH 8.5). Suitable buffers include phosphate buffer (e.g. PBS), bicarbonate buffer and/or Tris buffer. A typical composition is formulated in 1M saccharose, 150 mM NaCl, 1 mM MgCl2, 54 mg/1 Tween 80, 10 mM Tris pH 8.5. Another typical composition is formulated in 10 mg/ml mannitol, 1 mg/ml HSA, 20 mM Tris, pH 7.2, and 150 mM NaCl.

The composition of the disclosure can be in various forms, e.g. in solid (e.g. powder, lyophilized form), or liquid (e.g. aqueous). In the case of solid compositions, the typical methods of preparation are vacuum drying and freeze-drying which yields a powder of the active agent plus any additional desired ingredient from a previously sterile-filtered solution thereof. Such solutions can, if desired, be stored in a sterile ampoule ready for reconstitution by the addition of sterile water for ready injection.

Nebulized or aerosolized formulations also form part of this disclosure. Methods of intranasal administration are well known in the art, including the administration of a droplet, spray, or dry powdered form of the composition into the nasopharynx of the individual to be treated from a pressured container or dispenser which contains a suitable propellant, e.g., a gas such as carbon dioxide, or a nebulizer (see for example WO 95/11664). Enteric formulations such as gastroresistant capsules and granules for oral administration, suppositories for rectal or vaginal administration also form part of this disclosure. For non-parental administration, the compositions can also include absorption enhancers which increase the pore size of the mucosal membrane. Such absorption enhancers include sodium deoxycholate, sodium glycocholate, dimethyl-beta-cyclodextrin, lauroyl-1-lysophosphatidylcholine and other substances having structural similarities to the phospholipid domains of the mucosal membrane.

The composition can also contain other pharmaceutically acceptable excipients for providing desirable pharmaceutical or pharmacodynamic properties, including for example modifying or maintaining the pH, osmolarity, viscosity, clarity, color, sterility, stability, rate of dissolution of the formulation, modifying or maintaining release or absorption into an the human or animal organism. For example, polymers such as polyethylene glycol may be used to obtain desirable properties of solubility, stability, half-life and other pharmaceutically advantageous properties (Davis et al., 1978, Enzyme Eng. 4, 169-173; Burnham et al., 1994, Am. J. Hosp. Pharm. 51, 210-218). Representative examples of stabilizing components include polysorbate 80, L-arginine, polyvinylpyrrolidone, trehalose, and combinations thereof. Other stabilizing components especially suitable in plasmid-based compositions include hyaluronidase (which is thought to destabilize the extra cellular matrix of the host cells as described in WO 98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), nuclease inhibitors such as actin G (WO 99/56784) and cationic salts such as magnesium (Mg²⁺) (EP 998 945) and lithium (Li⁺) (WO 01/47563) and any of their derivatives. The amount of cationic salt in the composition of the disclosure typically ranges from about 0.1 mM to about 100 mM, and still more typically from about 0.1 mM to about 10 mM. Viscosity enhancing agents include sodium carboxymethylcellulose, sorbitol, and dextran. The composition can also contain substances known in the art to promote penetration or transport across the blood barrier or membrane of a particular organ (e.g. antibody to transferrin receptor; Friden et al., 1993, Science 259, 373-377). A gel complex of poly-lysine and lactose (Midoux et al., 1993, Nucleic Acid Res. 21, 871-878) or poloxamer 407 (Pastore, 1994, Circulation 90, 1-517) can be used to facilitate administration in arterial cells.

The composition of the disclosure may also comprise one or more adjuvant(s) suitable for systemic or mucosal application in humans. Representative examples of useful adjuvants include without limitation alum, mineral oil emulsion such as Freunds complete and incomplete, lipopolysaccharide or a derivative thereof (Ribi et al., 1986, Immunology and Immunopharmacology of Bacterial Endotoxins, Plenum Publ. Corp., NY, p407-419), saponins such as QS21 (Sumino et al., 1998, J. Virol. 72, 4931-4939; WO 98/56415), Escin, Digitonin, Gypsophila or Chenopodium quinoa saponins. Alternatively the composition of the disclosure may be formulated with conventional vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, and lipid-based particles, etc. The composition may also be formulated in the presence of cholesterol to form particulate structures such as liposomes.

The composition may be administered to patients in an amount effective, especially to enhance an immune response in an animal or human organism. As used herein, the term “effective amount” refers to an amount sufficient to realize a desired biological effect. For example, an effective amount for enhancing an immune response could be that amount necessary to cause activation of the immune system, for instance resulting in the development of an anti-tumor response in a cancerous patient (e.g. size reduction or regression of the tumor into which the composition has been injected and/or distant tumors). The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment is routinely made by a practitioner, in the light of the relevant circumstances. The titer may be determined by conventional techniques. A composition based on vector plasmids may be formulated in the form of doses of between 1 μg to 100 mg, advantageously between 10 μg and 10 mg and typically between 100 μg and 1 mg. A composition based on proteins may be formulated in the form of doses of between 10 ng to 100 mg. A typical dose is from about 1 μg to about 10 mg of the therapeutic protein per kg body weight. The administration may take place in a single dose or a dose repeated one or several times after a certain time interval. In one typical embodiment, the composition of the present disclosure is administered by injection using conventional syringes and needles, or devices designed for ballistic delivery of solid compositions (WO 99/27961), or needleless pressure liquid jet device (U.S. Pat. No. 4,596,556; U.S. Pat. No. 5,993,412).

The composition of the disclosure can be enclosed in ampoules, disposable syringes or multiple dose vials made of glass or plastic. In all cases, the composition must be sterile and should be fluid to the extent that easy syringability exists. It must be stable under the conditions of manufacture and storage and must be preserved against the contaminating action of microorganisms such as bacteria and fungi. Sterile injectable solutions can be prepared by incorporating the active agent (e.g., a fusion protein or infectious particles) in the required amount with one or a combination of ingredients enumerated above, followed by filtered sterilization.

### Methods of Use

The pharmaceutical composition of the disclosure may be employed in methods for treating or preventing a variety of diseases and pathologic conditions, including genetic diseases, congenital diseases and acquired diseases such as infectious diseases (e.g. viral and/or bacterial infections), cancer, immune deficiency diseases, and autoimmune diseases. Accordingly, the present disclosure also encompasses the use of the fusion protein, vector, infectious viral particle, host cell or composition of the disclosure for the preparation of a drug intended for treating or preventing such diseases, and especially cancer or an infectious disease.

Changes in mast cells tissue populations occurs during T helper 2 (Th2)-cell responses associated with inflammation and remodeling. Typically, the number of mast cells in the nasal cavity increases during allergy season. Th2 responses initiate increased numbers of circulating basophils, hematopoietic cells and secrete mediators such as histamine.

WBB6 F1-Kit^{W/W-v} and C57BL/6-Kit^{W-sh/W-sh} mice are deficient in mast cells. These mice can be selectively repaired for phenotypic abnormalities by the infusion of mast cells. The mice exhibit anemia, reduced numbers of bone-marrow and blood neutrophils, sterility, and markedly reduced numbers of interstitial cells of Cajal.

In certain embodiments, this disclosure relates to the use of GIFT9 for the treatment or prevention of anemia. In certain embodiments, this disclosure relates to methods of treating or preventing anemia comprising administering an effective amount of GIFT9 to a subject in need thereof.

The interstitial cell of Cajal (ICC) is a type of interstitial cell typically found in the gastrointestinal tract that serves as a pacemaker in smooth muscle. See Sanders et al., Annu Rev Physiol, 2006, 68: 307-343. Loss of ICC may interrupt normal neural control of gastrointestinal (GI) contractions and lead to functional GI disorders, such as irritable bowel syndrome.

Echtenacher et al., report a critical protective role of mast cells in a model of acute septic peritonitis. Nature. 1996; 381:75-77. In certain embodiment, this disclosure relates uses of GIFT9 for the treatment or prevention of GI disorders. In certain embodiments, this disclosure relates to methods of treating or preventing a GI disorder comprising administering an effective amount of GIFT9 to a subject in need thereof. In certain embodiments, the GI disorder is selected from peritonitis, irritable bowel syndrome, inflammatory bowel disease, Crohn's disease, ulcerative colitis, coeliac disease, parasitic infections, or small intestine bacterial overgrowth.

Waern et al., report mouse mast cell protease 4 is the major chymase in murine airways and has a protective role in allergic airway inflammation. See J Immunol, 2009, 183:6369-6376. In certain embodiment, this disclosure relates uses of GIFT9 for the treatment or prevention of allergic airway inflammation. In certain embodiments, this disclosure relates to methods of treating or preventing an allergic airway inflammation disorder or condition comprising administering an effective amount of GIFT9 to a subject in need thereof. In certain embodiments, the allergic airway inflammation is selected from asthma, emphysema, bronchitis, or chronic obstructive pulmonary disease.

McLachlan et al. report mast cell activators as a class of highly effective vaccine adjuvants. Nat Med. 2008; 14:536-541. Malaviya et al., report mast cell modulation of neutrophil influx and bacterial clearance at sites of infection through TNF-alpha. See Nature, 1996, 381:77-80. Dawicki & Marshall report roles for mast cells in host defense. Curr Opin Immunol, 2007, 19:31-38. Metz et al. report mast cell functions in the innate skin immune system. Immunobiology, 2008, 213:251-260. Matsuda report the necessity of IgE antibodies and mast cells for manifestation of resistance against larval Haemaphysalis longicornis ticks in mice. See J Immunol. 1990, 144:259-262. Maurer et al., report skin mast cells control T cell-dependent host defense in Leishmania major infections. Faseb J, 2006, 20:2460-2467. Furuta et al., report protective roles of mast cells and mast cell-derived TNF in murine malaria. J Immunol, 2006, 177:3294-3302. Abe & Nawa report reconstitution of mucosal mast cells in W/W^{V} mice by adoptive transfer of bone marrow-derived cultured mast cells and its effects on the protective capacity to Strongyloides ratti infection. See Parasite Immunol, 1987, 9:31-38.

In certain embodiments, this disclosure relates to methods of treating or preventing a pathogenic infection or associated condition comprising administering an effective amount of GIFT9 to a subject in need thereof. In certain embodiments, infectious diseases associated is with an infection by a pathogen such as fungi, bacteria, protozoa, tick, malaria, and viruses.

Representative examples of viral pathogens include without limitation human immunodeficiency virus (e.g. HIV-1 or HIV-2), human herpes viruses (e.g. HSV1 or HSV2), cytomegalovirus, Rotavirus, Epstein Barr virus (EBV), hepatitis virus (e.g. hepatitis B virus, hepatitis A virus, hepatitis C virus and hepatitis E virus), varicella-zoster virus (VZV), paramyxoviruses, coronaviruses; respiratory syncytial virus, parainfluenza virus, measles virus, mumps virus, flaviviruses (e.g. Yellow Fever Virus, Dengue Virus, Tick-borne encephalitis virus, Japanese Encephalitis Virus), influenza virus, and typically human papilloma viruses (e.g. HPV-6, 11, 16, 18, 31. 33). Representative examples of bacterial pathogens include Neisseria (e.g. N. gonorrhea and N. meningitidis); Bordetella (e.g. B. pertussis, B. parapertussis and B. bronchiseptica), Mycobacteria (e.g. M. tuberculosis, M. bovis, M. leprae, M. avium, M. paratuberculosis, M. smegmatis); Legionella (e.g. L. pneumophila); Escherichia (e.g. enterotoxic E. coli, enterohemorragic E. coli, enteropathogenic E. coli); Vibrio (e.g. V. cholera); Shigella (e.g. S. sonnei, S. dysenteriae, S. flexnerii); Salmonella (e.g. S. typhi, S. paratyphi, S. choleraesuis, S. enteritidis); Listeria(e.g. L. monocytogenes); Helicobacter (e.g. H. pylori); Pseudomonas (e.g. P. aeruginosa); Staphylococcus (e.g. S. aureus, S. epidermidis); Enterococcus (e.g. E. faecalis, E. faecium), Clostridium (e.g. C. tetani, C. botulinum, C. difficile); Bacillus (e.g. B. anthracis); Corynebacterium (e.g. C. diphtheriae), and Chlamydia (e.g. C. trachomatis, C. pneumoniae, C. psittaci). Representative examples of parasite pathogens include Plasmodium (e.g. P. falciparum), Toxoplasma (e.g. T. gondii) Leshmania (e.g. L. major), Pneumocystis (e.g. P. carinii), Trichomonas (e.g. T. vaginalis), Schisostoma (e.g. S. mansoni). Representative examples of fungi include Candida (e.g. C. albicans) and Aspergillus.

In certain embodiments, the disclosure contemplates the use of GIFT9 as a vaccine adjuvant. In certain embodiments, the disclosure relates to methods of preventing an infection comprising administering an effective amount of GIFT9 with an adjuvant or a vaccine.

In certain embodiments, the bacteria is selected from the group consisting of Acinetobacter baumannii, Bordetella pertussis, Burkholderia cepacia, Burkholderia pseudomallei, Burkholderia mallei, Campylobacter jejuni, Campylobacter coli, Enterobacter cloacae, Enterobacter aerogenes, Escherichia coli, Francisella tularensis, Haemophilus influenzae, Haemophilus ducreyi, Helicobacter pylori, Klebsiella pneumoniae, Legionella penumophila, Moraxella catarrhalis, Morganella morganii, Neisseria gonorrhoeae, Neisseria meningitidis, Pasteurella multocida, Proteus mirabilis, Proteus vulgaris, Pseudomonas aeruginosa, Salmonella typhi, Serratia marcescens, Shigella flexneri, Shigella boydii, Shigella sonnei, Shigella dysenteriae, Stenotrophomonas maltophilia, Vibrio cholerae, and Chlamydia pneumoniae.

The composition of the present disclosure is particularly intended for the preventive or curative treatment of disorders, conditions or diseases associated with cancer. The term “cancer” encompasses any cancerous conditions including diffuse or localized tumors, metastasis, cancerous polyps and preneoplastic lesions (e.g. dysplasies) as well as diseases which result from unwanted cell proliferation. A variety of tumors may be selected for treatment in accordance with the methods described herein. In general, solid tumors are typical. Cancers which are contemplated in the context of the disclosure include without limitation glioblastoma, sarcoma, melanomas, mastocytoma, carcinomas as well as breast cancer, prostate cancer, testicular cancer, ovarian cancer, endometrial cancer, cervical cancer (in particular, those induced by a papilloma virus), lung cancer (e.g. lung carcinomas including large cell, small cell, squamous and adeno-carcinomas), renal cancer, bladder cancer, liver cancer, colon cancer, anal cancer, pancreatic cancer, stomach cancer, gastrointestinal cancer, cancer of the oral cavity, larynx cancer, brain and CNS cancer, skin cancer (e.g. melanoma and non-melanoma), blood cancer (lymphomas, leukemia, especially if they have developed in solid mass), bone cancer, retinoblastoma and thyroid cancer. In one typical embodiment of the use of the disclosure, the composition is administered into or in close proximity to a solid tumor.

In certain embodiments, the disclosure contemplates uses of conjugates disclosed herein in autologous immune enhancement therapy (AIET). AIET is a treatment method in which immune or cancer cells, e.g., lymphokine-activated killer (LAK) cells, natural killer (NK) cells, cytotoxic T lymphocytes (CTLs), dendritic cells (DCs), are taken out from the body of a subject which are cultured and processed to activate them until their resistance to cancer is strengthened and then the cells are put back in the body. The cells, antibodies, and organs of the immune system work to protect and defend the body against the tumor cells. In certain embodiments, the disclosure contemplates mixing harvested cells with conjugates of GM-CSF and IL-9 to activate them. In certain embodiments, the disclosure contemplates administering conjugates of GM-CSF and IL-9 when the cells are administered back to the subject.

In certain embodiments, the disclosure contemplates the administration of sipuleucel-T (PROVENGE) in combination with a conjugate of GM-CSF and IL-9. PROVENGE consists of autologous peripheral blood mononuclear cells, including antigen presenting cells (APCs), that have been activated during a culture period with a recombinant human protein, PAP-GM-CSF, consisting of prostatic acid phosphatase (PAP), an antigen expressed in prostate cancer tissue, linked to GM-CSF. In certain embodiments, the disclosure relates to a conjugate comprising PAP, GM-CSF, and IL-9, and uses in activating antigen presenting cells in peripheral blood mononuclear cells. The peripheral blood mononuclear cells of the subject may be obtained via a standard leukapheresis procedure prior to infusion. During culture, the recombinant antigen can bind to and be processed by antigen presenting cells (APCs). The recombinant antigen is believed to direct the immune response to PAP. The infused product is believed to contain antigen presenting cells, dendritic cells, T cells, B cells, natural killer (NK) cells, and other cells. Typically each dose contains more than 50 million autologous CD54⁺ cells activated with PAP-GM-CSF or PAP-GM-CSF-IL-9. The potency is typically evaluated by measuring the increased expression of the CD54 molecule, also known as ICAM-1, on the surface of APCs after culture with PAP-GM-CSF or PAP-CM-CSF-IL-9. CD54 is a cell surface molecule that plays a role in the immunologic interactions between APCs and T cells, and is considered a marker of immune cell activation.

In certain embodiments, the disclosure contemplates methods for treating cancer comprising administering any GM-CSF and IL-9 conjugate disclosed herein as an immune adjuvant in combination with a vector that encodes a tumor associated antigen/cancer marker, such as PSA, PAP, and optionally encoding other co-stimulatory molecules selected from, B7-1, B7-2, ICAM-1, GM-CSF, leukocyte function-associated antigen-3 (LFA-3). Other embodiments contemplated for the treatment of cancer include administering an effective amount of a vector that encodes a GM-CSF and IL-9 conjugate disclosed herein and optionally further encodes a tumor associated antigen/cancer marker and optionally encodes other co-stimulatory molecules to a subject. PROSTVAC is a recombinant vector encoding costimulatory molecules, as well as PSA as a vaccine target. Plasmid DNA is incorporated into either vaccinia or fowlpox viruses by means of a packing cell line. Patients are treated with a vaccinia prime followed by a series of fowlpox-based boosts.

In certain embodiments, the disclosure relates to methods of treating cancer comprising administering a GM-CSF and IL-9 conjugate in combination with an anti-CTLA-4 antibody. Anti-CTLA-4 antibody is contemplated to be administered in combination with any of the methods disclosed herein. It is believed that it binds to CTLA-4 surface glycoprotein on T-cell surface, minimizing immune autoregulation and potentially enhancing antitumor activity. Interactions between B7 molecules on antigen-presenting cells and CTLA-4 on tumor-specific T cells are inhibitory. Thus, CTLA-4 engagement negatively regulates the proliferation and function of such T cells. Under certain conditions, blocking CTLA-4 with a monoclonal antibody (ipilimumab or tremilimumab) restores T-cell function.

Other embodiments contemplated for the treatment of cancer include methods that utilize the extraction of cancer cells from a subject and incorporate glycosyl-phosphatidylinositol (GPI)-anchored co-stimulatory molecules such as B7-1 and B7-2 into tumor cell membranes optionally with a conjugate GM-CSF and IL-9 anchored GPI, and administering the modified cells to the subject in combination with a conjugate of GM-CSF and IL-9 to elicit an immune response. See e.g., McHugh et al., Cancer Res., 1999, 59 (10):2433-7; Poloso et al., Mol Immunol., 2002, 38 (11):803-16; and Nagarajan & Selvaraj, Cancer Res., 2002, 62 (10):2869-74.

Examples of autoimmune diseases include, but are not limited to, multiple sclerosis (MS), scleroderma, rheumatoid arthritis, autoimmune hepatitis, diabetes mellitus, ulcerative colitis, Myasthenia gravis, systemic lupus erythematosus, Graves' disease, idiopathic thrombocytopenia purpura, hemolytic anemia, multiple myositis/dermatomyositis, Hashimoto's disease, autoimmune hypocytosis, Sjogren's syndrome, angitis syndrome and drug-induced autoimmune diseases (e.g., drug-induced lupus).

Moreover, as mentioned above, the fusion protein, nucleic acid molecule, vector, infectious particle, host cell and/or composition of the present disclosure can be used as an adjuvant to enhance the immune response of an animal or human organism to a particular antigen. This particular use of the present disclosure may be made in combination with one or more transgenes or transgene products as defined above, e.g. for purposes of immunotherapy. Typically, the active agent (e.g. fusion protein, infectious particle or pharmaceutical composition of the disclosure) is administered in combination with one or more transgenes or transgene products. Accordingly, there is typically also provided a composition comprising in combination a transgene product (e.g. a viral antigen or a suicide gene product) and a fusion protein as well as a composition comprising vector(s) or viral particles encoding a transgene product and a fusion protein. The transgene and the fusion-encoding nucleic acid sequences may be expressed from the same vector or from separate vectors which may have the same origin (e.g. adenoviral vectors) or a different origin (e.g. a MVA vector encoding the particular antigen and an adenoviral vector encoding the fusion protein). The fusion protein and the transgene product (or their respective encoding vectors) can be introduced into the host cell or organism either concomitantly or sequentially either via the mucosal and/or systemic route.

### Combination Therapies

In certain embodiments, this disclosure relates to methods of administrating effective amounts of GIFT9 in combination with exposure to an antigen or administration of a vaccine. The antigen can be any type of antigen against which an immune response is desired in a subject, or any antigen to which a subject is exposed. In some cases, a subject is exposed to the antigen during an infection, such as a viral, bacterial, fungal or parasitic infection. In other cases, the subject has a tumor and is exposed to a tumor-specific antigen. Alternatively, the antigen can be administered to a subject, such as in the form of a vaccine. In some embodiments, the vaccine is a vaccine against a pathogen, or a cancer vaccine.

In some embodiments, the antigen is an antigen from a pathogen, such as a virus, bacterium, fungus or parasite. Viral pathogens include, but are not limited to retroviruses, such as human immunodeficiency virus (HIV) and human T-cell leukemia viruses; picornaviruses, such as polio virus, hepatitis A virus; hepatitis C virus, enteroviruses, human coxsackie viruses, rhinoviruses, echoviruses, and foot-and-mouth disease virus; caliciviruses, such as strains that cause gastroenteritis (e.g., Norwalk virus); togaviruses, such as alphaviruses (including chikungunya virus, equine encephalitis viruses, Sindbis virus, Semliki Forest virus, and Ross River virus) and rubella virus; flaviviruses, such as dengue viruses, yellow fever viruses, West Nile virus, St. Louis encephalitis virus, Japanese encephalitis virus, Powassan virus and other encephalitis viruses; coronaviruses, including severe acute respiratory syndrome (SARS) virus; rhabdoviruses, such as vesicular stomatitis virus and rabies virus; filoviruses, such as Ebola virus and Marburg virus); paramyxoviruses, such as parainfluenza virus, mumps virus, measles virus, and respiratory syncytial virus; orthomyxoviruses, such as influenza viruses (including avian influenza viruses and swine influenza viruses); bunyaviruses, such as Hantaan virus; Sin Nombre virus, and Rift Valley fever virus, phleboviruses and Nairo viruses; arenaviruses, such as Lassa fever virus and other hemorrhagic fever viruses, Machupo virus and Junin virus; reoviruses, such as mammalian reoviruses, orbiviurses and rotaviruses; birnaviruses; hepadnaviruses, such as hepatitis B virus; parvoviruses; papovaviruses, such as papilloma viruses, polyoma viruses and BK-virus; adenoviruses; herpesviruses, such as herpes simplex virus (HSV)-1 and HSV-2, cytomegalovirus, Epstein-Barr virus, varicella zoster virus, and other herpes viruses, including HSV-6); pox viruses, such as variola viruses and vaccinia viruses; irodoviruses, such as African swine fever virus; astroviruses; and unclassified viruses (for example, the etiological agents of spongiform encephalopathies, the agent of delta hepatitis (thought to be a defective satellite of hepatitis B virus).

Bacterial pathogens include, but are not limited to Helicobacter pylori, Escherichia coli, Vibrio cholerae, Borelia burgdorferi, Legionella pneumophilia, Mycobacteria sps (such as. M. tuberculosis, M. avium, M. intracellulare, M. kansai and, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Bordetella pertussis, Shigella flexnerii, Shigella dysenteriae and Actinomyces israelli.

Fungal pathogens include, but are not limited to Cryptococcus neoformans, Histoplasma capsulatum, Coccidioides immitis, Blastomyces dermatitidis, Chlamydia trachomatis, Candida albicans. Parasitic pathogens include, but are not limited to Plasmodium falciparum, Plasmodium vivax, Trypanosoma cruzi and Toxoplasma gondii.

In some embodiments, a subject is administered GIFT9 following diagnosis of the subject (e.g. a diagnosis of the presence of an infection or cancer). The GIFT9 can be administered in a single dose or in multiple doses over time. In some examples, a subject having an infection or cancer is administered GIFT9 daily for at least one week, at least one month or at least three months.

In some embodiments, GIFT9 is administered in combination with a vaccine e.g., live or attenuated virus. A number of vaccines against infectious diseases have been approved for use in the United States, contemplated examples include Anthrax Vaccine, BIOTHRAX™, BCG Vaccine, TICE BCG™, BCG Vaccine, MYCOBAX™, Diphtheria & Tetanus Toxoids, TRIPEDIA™, INFANRIX™, DAPTACEL™, PEDIARIX™, Diphtheria and Tetanus Toxoids and Acellular Pertussis and Inactivated Poliovirus Vaccine, KINRIX™, Diphtheria and Tetanus Toxoids and Acellular Pertussis, Inactivated Poliovirus and Haemophilus b Conjugate (Tetanus Toxoid Conjugate) Vaccine, PENTACEL™Haemophilus b Conjugate Vaccine (Diphtheria CRM197 Protein Conjugate), HIBTITER™Haemophilus b Conjugate Vaccine (Meningococcal Protein Conjugate), PEDVAXHIB™Haemophilus b Conjugate Vaccine (Tetanus Toxoid Conjugate) ACTHIB™, Haemophilus b Conjugate Vaccine (Meningococcal Protein Conjugate) & Hepatitis B Vaccine (Recombinant), COMVAX™, Hepatitis A Vaccine, HAVRIX™, VAQTA™, Hepatitis A Inactivated and Hepatitis B (Recombinant) Vaccine, TWINRIX™, ENGERIX-B™ Hepatitis B Vaccine (Recombinant) RECOMBIVAX HB™, Human Papillomavirus (Types 6, 11, 16, 18) Recombinant Vaccine, GARDASIL™, Influenza Virus Vaccine, AFLURIA™, Influenza Virus Vaccine, H5N1, Influenza Virus Vaccine, Trivalent, Types A and B, FLULAVAL™, Influenza Virus Vaccine, FLUMIST™, FLUARIX™, FLUVIRIN™, FLUZONE™, Japanese Encephalitis Virus Vaccine, JE-VAX™, Measles Virus Vaccine, ATTENUVAX™, Measles and Mumps Virus Vaccine, M-M-Vax™, Measles, Mumps, and Rubella Virus Vaccine, M-M-R II™, Measles, Mumps, Rubella and Varicella Virus Vaccine, PROQUAD™, Meningococcal Polysaccharide (Serogroups A, C, Y and W-135) Diphtheria Toxoid Conjugate Vaccine, MENACTRA™, Meningococcal Polysaccharide Vaccine, Groups A, C, Y and W-135 Combined, MENOMUNE-A/C/Y/W-135™, Mumps Virus Vaccine, MUMPSVAX™, Plague Vaccine, Pneumococcal Vaccine, PNEUMOVAX 23™, Pneumococcal 7-valent Conjugate Vaccine (Diphtheria CRM197 Protein), PREVNAR™, Poliovirus Vaccine Inactivated (Human Diploid Cell), POLIOVAX™, Poliovirus Vaccine (Monkey Kidney Cell), IPOL™, Rabies Vaccine, IMOVAX™, RABAVERT™, Rotavirus Vaccine, ROTARIX™, ROTATEQ™, Rubella Virus Vaccine, MERUVAX II™, Smallpox (Vaccinia) Vaccine, ACAM2000™, DRYVAX™, Tetanus & Diphtheria Toxoids, DECAVAC™, TENIVAC™, Tetanus Toxoid, Tetanus Toxoid, Reduced Diphtheria Toxoid and Acellular Pertussis Vaccine, ADACEL™, Tetanus Toxoid, Reduced Diphtheria Toxoid and Acellular Pertussis Vaccine, BOOSTRIX™, Typhoid Vaccine Ty21a, VIVOTIF™, Typhoid Vi Polysaccharide Vaccine, TYPHIM VI™, Varicella Virus Vaccine, VARIVAX™, Yellow Fever Vaccine, YF-VAX™, and Zoster Vaccine, ZOSTAVAX™.

In some cases, the antigen is a tumor-associated antigen. Tumor antigens are proteins that are produced by tumor cells that elicit an immune response, particularly T-cell mediated immune responses. The tumor antigen can be any tumor-associated antigen, which are well known in the art and include, for example, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, macrophage colony stimulating factor, prostase, prostate-specific antigen (PSA), PAP, NY-ESO-1, LAGE-1a, p53, prostein, PSMA, Her2/neu, survivin and telomerase, prostate-carcinoma tumor antigen-1, MAGE, ELF2M, neutrophil elastase, ephrinB2, CD22, insulin growth factor (IGF)-I, IGF-II, IGF-I receptor and mesothelin.

The cancer treatments disclosed herein can be applied as a sole therapy or can involve, conventional surgery or radiotherapy, hormonal therapy, or chemotherapy. Such chemotherapy can include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-Her2 antibody trastuzumab and the anti-epidermal growth factor receptor (EGFR) antibody, cetuximab), farnesyl transferase inhibitors, tyrosine kinase inhibitors and serine/threonine kinase inhibitors, for example inhibitors of the epidermal growth factor family for example EGFR family tyrosine kinase inhibitors such as: N-(3-chloro-4-fluorophenyl)-7-methoxy-6-(3-morpholinopropoxy)quinazolin-4-a mine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors of phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (Abl) tyrosine kinase family such as dasatinib (BMS-354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvβ3 function and angiostatin);

(vi) vascular damaging agents such as Combretastatin A4;

(vii) antisense therapies, for example those which are directed to the targets listed above, such as an anti-RAS antisense; and

(viii) immunotherapy approaches, including for example ex-vivo and in-vivo approaches to increase the immunogenicity of subject tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

The combination therapy also contemplates use of the disclosed pharmaceutical compositions with radiation therapy or surgery, as an alternative, or a supplement, to a second therapeutic or chemotherapeutic agent.

A typical chronic lymphocytic leukemia (CLL) chemotherapeutic plan includes combination chemotherapy with chlorambucil or cyclophosphamide, plus a corticosteroid such as prednisone or prednisolone. The use of a corticosteroid has the additional benefit of suppressing some related autoimmune diseases, such as immunohemolytic anemia or immune-mediated thrombocytopenia. In resistant cases, single-agent treatments with nucleoside drugs such as fludarabine, pentostatin, or cladribine may be successful. Patients may consider allogeneic or autologous bone marrow transplantation. In certain embodiments, the disclosure contemplates combination treatments using conjugates disclosed herein in combination with chloroambucil, cyclophosphamide, prednisone, prednisolone, fludarabine, pentostatin, and/or cladribine or combinations thereof.

Treatment of acute lymphoblastic leukemia typically includes chemotherapy to bring about bone marrow remission. Typical regiments include prednisone, vincristine, and an anthracycline drug, L-asparaginase or cyclophosphamide. Other options include tprednisone, L-asparaginase, and vincristine. Consolidation therapy or intensification therapy to eliminate any remaining leukemia may include antimetabolite drugs such as methotrexate and 6-mercaptopurine (6-MP). In certain embodiments, the disclosure contemplates combination treatments using conjugates disclosed herein in combination with COP, CHOP, R-CHOP, imatinib, alemtuzumab, vincristine, L-asparaginase or cyclophosphamide, methotrexate and/or 6-mercaptopurine (6-MP). COP refers to a chemotherapy regimen used in the treatment of lymphoma of cyclophosphamide, vincristine, and prednisone or prednisolone and optionally hydroxydaunorubicin (CHOP) and optionally rituximab (R-CHOP).

In some embodiments, the disclosure relates to treating a viral infection by administering a GM-CSF and IL-9 conjugate in combination with a second antiviral agent. In further embodiments, a GM-CSF and IL-9 conjugate is administered in combination with one or more of the following agents: abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir (Tamiflu), peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir (Valtrex), valganciclovir, vicriviroc, vidarabine, viramidine zalcitabine, zanamivir (Relenza), and/or zidovudine (AZT).

Antiviral agents include, but are not limited to, protease inhibitors (PIs), integrase inhibitors, entry inhibitors (fusion inhibitors), maturation inhibitors, and reverse transcriptase inhibitors (anti-retrovirals). Combinations of antiviral agents create multiple obstacles to viral replication, i.e., to keep the number of offspring low and reduce the possibility of a superior mutation. If a mutation that conveys resistance to one of the agents being taken arises, the other agents continue to suppress reproduction of that mutation. For example, a single anti-retroviral agent has not been demonstrated to suppress an HIV infection for long. These agents are typically taken in combinations in order to have a lasting effect. As a result, the standard of care is to use combinations of anti-retrovirals.

Reverse transcribing viruses replicate using reverse transcription, i.e., the formation of DNA from an RNA template. Retroviruses often integrate the DNA produced by reverse transcription into the host genome. They are susceptible to antiviral drugs that inhibit the reverse transcriptase enzyme. In certain embodiments the disclosure relates to methods of treating viral infections by administering a GM-CSF and IL-9 conjugate, and a retroviral agent such as nucleoside and nucleotide reverse transcriptase inhibitors (NRTI) and/or a non-nucleoside reverse transcriptase inhibitors (NNRTI). Examples of nucleoside reverse transcriptase inhibitors include zidovudine, didanosine, zalcitabine, stavudine, lamivudine, abacavir, emtricitabine, entecavir, apricitabine. Examples of nucleotide reverse transcriptase inhibitors include tenofovir and adefovir. Examples of non-nucleoside reverse transcriptase inhibitors include efavirenz, nevirapine, delavirdine, and etravirine.

In certain embodiments, the disclosure relates to methods of treating a viral infection by administering a GM-CSF and IL-9 conjugate in combination with an antiviral drug, e.g., 2′,3′-dideoxyinosine and a cytostatic agent, e.g., hydroxyurea.

Human immunoglobulin G (IgG) antibodies are believed to have opsonizing and neutralizing effects against certain viruses. IgG is sometimes administered to a subject diagnosed with immune thrombocytopenic purpura (ITP) secondary to a viral infection since certain viruses such as, HIV and hepatitis, cause ITP. In certain embodiments, the disclosure relates to methods of treating or preventing viral infections comprising administering a GM-CSF and IL-9 conjugate in combination with an immunoglobulin to a subject. IgG is typically manufactured from large pools of human plasma that are screened to reduce the risk of undesired virus transmission. The Fc and Fab functions of the IgG molecule are usually retained. Therapeutic IgGs include Privigen, Hizentra, and WinRho. WinRho is an immunoglobulin (IgG) fraction containing antibodies to the Rho(D) antigen (D antigen). The antibodies have been shown to increase platelet counts in Rho(D) positive subjects with ITP. The mechanism is thought to be due to the formation of anti-Rho(D) (anti-D)-coated RBC complexes resulting in Fc receptor blockade, thus sparing antibody-coated platelets.

In some embodiments, the disclosure relates to treating a bacterial infection by administering a GM-CSF and IL-9 conjugate in combination with an antibiotic drug. In further embodiments, the subject is co-administered with an antibiotic selected from the group comprising of Sulfonamides, Diaminopyrimidines, Quinolones, Beta-lactam antibiotics, Cephalosporins, Tetracyclines, Notribenzene derivatives, Aminoglycosides, Macrolide antibiotics, Polypeptide antibiotics, Nitrofuran derivatives, Nitroimidazoles, Nicotinin acid derivatives, Polyene antibiotics, Imidazole derivatives or Glycopeptide, Cyclic lipopeptides, Glycylcyclines and Oxazolidinones. In further embodiments, these antibiotics include but are not limited to Sulphadiazine, Sulfones—[Dapsone (DDS) and Paraaminosalicyclic (PAS)], Sulfanilamide, Sulfamethizole, Sulfamethoxazole, Sulfapyridine, Trimethoprim, Pyrimethamine, Nalidixic acids, Norfloxacin, Ciproflaxin, Cinoxacin, Enoxacin, Gatifloxacin, Gemifloxacin, Grepafloxacin, Levofloxacin, Lomefloxacin, Moxifloxacin, Ofloxacin, Pefloxacin, Sparfloxacin, Trovafloxacin, Penicillins (Amoxicillin, Ampicillin, Azlocillin, Carbenicillin, Cloxacillin, Dicloxacillin, Flucloxacillin, Hetacillin, Oxacillin, Mezlocillin, Penicillin G, Penicillin V, Piperacillin), Cephalosporins (Cefacetrile, Cefadroxil, Cefalexin, Cefaloglycin, Cefalonium, Cefaloridin, Cefalotin, Cefapirin, Cefatrizine, Cefazaflur, Cefazedone, Cefazolin, Cefradine, Cefroxadine, Ceftezole, Cefaclor, Cefonicid, Ceforanide, Cefprozil, Cefuroxime, Cefuzonam, Cefmetazole, Cefoteta, Cefoxitin, Cefcapene, Cefdaloxime, Cefdinir, Cefditoren, Cefetamet, Cefixime, Cefmenoxime, Cefodizime, Cefoperazone, Cefotaxime, Cefotiam, Cefpimizole, Cefpiramide, Cefpodoxime, Cefteram, Ceftibuten, Ceftiofur, Ceftiolen, Ceftizoxime, Ceftriaxone, Cefoperazone, Ceftazidime, Cefepime), Moxolactam, Carbapenems (Imipenem, Ertapenem, Meropenem) Monobactams (Aztreonam) Oxytetracycline, Chlortetracycline, Clomocycline, Demeclocycline, Tetracycline, Doxycycline, Lymecycline, Meclocycline, Methacycline, Minocycline, Rolitetracycline, Chloramphenicol, Amikacin, Gentamicin, Framycetin, Kanamycin, Neomicin, Neomycin, Netilmicin, Streptomycin, Tobramycin, Azithromycin, Clarithromycin, Dirithromycin, Erythromycin, Roxithromycin, Telithromycin, Polymyxin-B, Colistin, Bacitracin, Tyrothricin Notrifurantoin, Furazolidone, Metronidazole, Tinidazole, Isoniazid, Pyrazinamide, Ethionamide, Nystatin, Amphotericin-B, Hamycin, Miconazole, Clotrimazole, Ketoconazole, Fluconazole, Rifampacin, Lincomycin, Clindamycin, Spectinomycin, Chloramphenicol, Clindamycin, Colistin, Fosfomycin, Loracarbef, Metronidazole, Nitrofurantoin, Polymyxin B, Polymyxin B Sulfate, Procain, Spectinomycin, Tinidazole, Trimethoprim, Ramoplanin, Teicoplanin, Vancomycin, Trimethoprim, Sulfamethoxazole, and/or Nitrofurantoin.

### Vectors

In certain embodiments, the disclosure relates to expression system configure to produce GM-CSF/IL-9 conjugate comprising a recombinant vector disclosed herein. The term “vector” as used herein refers to both expression and non-expression vectors and includes viral as well as non-viral vectors, including autonomous self-replicating circular plasmids. Where a recombinant microorganism or cell culture is described as hosting an “expression vector,” this includes both extra-chromosomal circular DNA and DNA that has been incorporated into the host chromosome(s). Typical vectors of the disclosure are expression vectors. An expression vector contains multiple genetic elements positionally and sequentially oriented, i.e., operatively linked with other necessary elements such that nucleic acid molecule in the vector encoding the fusion proteins of the disclosure can be transcribed, and when necessary, translated in the host cells.

Any type of vector can be used in the context of the present disclosure, whether of plasmid or viral origin, whether it is an integrating or non-integrating vector. Such vectors are commercially available or described in the literature. Contemplated in the context of the disclosure are vectors for use in gene therapy (i.e. which are capable of delivering the nucleic acid molecule to a target cell) as well as expression vectors for use in recombinant techniques (i.e. which are capable for example of expressing the nucleic acid molecule of the disclosure in cultured host cells).

The vectors of the disclosure can function in prokaryotic or eukaryotic cells or in both (shuttle vectors). Suitable vectors include without limitation vectors derived from bacterial plasmids, bacteriophages, yeast episomes, artificial chromosomes, such as BAC, PAC, YAC, or MAC, and vectors derived from viruses such as baculoviruses, papovaviruses (e.g. SV40), herpes viruses, adenoviruses, adenovirus-associated viruses (AAV), poxviruses, foamy viruses, and retroviruses. Vectors may also be derived from combinations of these sources such as those derived from plasmid and bacteriophage genetic elements, e.g. cosmids and phagemids. Viral vectors can be replication-competent, conditionally replicative or replication-defective. In the case in which viral replication is defective, replication will occur in host cells providing functions that complement the defects.

Examples of suitable plasmids include but are not limited to those derived from pBR322 (Gibco BRL), pUC (Gibco BRL), pBluescript (Stratagene), p Poly (Lathe et al., 1987, Gene 57, 193-201), pTrc (Amann et al., 1988, Gene 69, 301-315) and pET 11d (Studier et al., 1990, Gene Expression Technology: Methods in Enzymology 185, 60-89). It is well known that the four of the plasmid can affect the expression efficiency, and it is typical that a large fraction of the vector be in supercoiled form. Examples of vectors for expression in yeast (e.g. S. cerevisiae) include pYepSec1 (Baldari et al., 1987, EMBO J. 6, 229-234), pMFa (Kujan et al., 1982, Cell 30, 933-943), pJRY88 (Schultz et al., 1987, Gene 54, 113-123), and pYES2 (Invitrogen Corporation, San Diego, Calif.). The vectors of the disclosure can also be derived from baculoviruses to be expressed in cultured insect cells (e.g. Sf 9 cells).

According to a typical embodiment of the disclosure, the nucleic acid molecules described herein are expressed by using mammalian expression vectors. Examples of mammalian expression vectors include pREP4, pCEP4 (Invitrogene), pCI (Promega), pCDM8 (Seed, 1987, Nature 329, 840) and pMT2PC (Kaufman et al., 1987, EMBO J. 6, 187-195). The expression vectors listed herein are provided by way of example only of some well-known vectors available to those of ordinary skill in the art. The person of ordinary skill in the art would be aware of other vectors suitable for maintenance, propagation or expression of the nucleic acid molecules described herein.

Moreover, the vector of the present disclosure may also comprise a marker gene in order to select or to identify the transfected cells (e.g. by complementation of a cell auxotrophy or by antibiotic resistance), stabilizing elements (e.g. cer sequence; Summers and Sherrat, 1984, Cell 36, 1097-1103), integrative elements (e.g. LTR viral sequences and transposons) as well as elements providing a self-replicating function and enabling the vector to be stably maintained in cells, independently of the copy number of the vector in the cell. Markers include tetracycline or ampicillin-resistance genes for prokaryotic host cells and dihydrofolate reductase or neomycin resistance for eukaryotic host cells. However, any marker that provides selection for a phenotypic trait will be effective. The self-replicating function may be provided by using a viral origin of replication and providing one or more viral replication factors that are required for replication mediated by that particular viral origin (WO 95/32299). Origins of replication and any replication factors may be obtained from a variety of viruses, including Epstein-Barr virus (EBV), human and bovine papilloma viruses and papovavirus BK.

Typical vectors of the present disclosure are viral vectors and especially adenoviral vectors, which have a number of well-documented advantages as vectors for gene therapy. The adenoviral genome consists of a linear double-stranded DNA molecule of approximately 36 kb carrying more than about thirty genes necessary to complete the viral cycle. The early genes are divided into 4 regions (E1 to E4) that are essential for viral replication (Pettersson and Roberts, 1986, In Cancer Cells (Vol 4): DNA Tumor Viruses, Botchan and Glodzicker Sharp Eds pp 37-47, Cold Spring Harbor Laboratory, Cold Spring Harbor, N.Y.; Halbert et al., 1985, J. Virol. 56, 250-257) with the exception of the E3 region, which is believed dispensable for viral replication based on the observation that naturally-occurring mutants or hybrid viruses deleted within the E3 region still replicate like wild-type viruses in cultured cells (Kelly and Lewis, 1973, J. Virol. 12, 643-652). The E1 gene products encode proteins responsible for the regulation of transcription of the viral genome. The E2 gene products are required for initiation and chain elongation in viral DNA synthesis. The proteins encoded by the E3 prevent cytolysis by cytotoxic T cells and tumor necrosis factor (Wold and Gooding, 1991, Virology 184, 1-8). The proteins encoded by the E4 region are involved in DNA replication, late gene expression and splicing and host cell shut off (Halbert et al., 1985, J. Virol. 56, 250-257). The late genes (L1 to L5) encode in their majority the structural proteins constituting the viral capsid. They overlap at least in part with the early transcription units and are transcribed from a unique promoter (MLP for Major Late Promoter). In addition, the adenoviral genome carries at both extremities cis-acting 5′ and 3′ ITRs (Inverted Terminal Repeat) and the encapsidation region, both essential for DNA replication. The ITRs harbor origins of DNA replication whereas the encapsidation region is required for the packaging of adenoviral DNA into infectious particles.

The adenoviral vectors for use in accordance with the present disclosure, typically infects mamailain cells. It can be derived from any human or animal source, in particular canine (e.g. CAV-1 or CAV-2; Genbank ref CAV1GENOM and CAV77082 respectively), avian (Genbank ref AAVEDSDNA), bovine (such as BAV3; Seshidhar Reddy et al., 1998, J. Virol. 72, 1394-1402), murine (Genbank ref ADRMUSMAV1), ovine, feline, porcine or simian adenovirus or alternatively from a hybrid thereof. Any serotype can be employed from the adenovirus serotypes 1 through 51. For instance, an adenovirus can be of subgroup A (e.g. serotypes 12, 18, and 31), subgroup B (e.g. serotypes 3, 7, 11, 14, 16, 21, 34, and 35), subgroup C (e.g. serotypes 1, 2, 5, and 6), subgroup D (e.g. serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. However, the human adenoviruses of the B or C sub-group are typical and especially adenoviruses 2 (Ad2), 5 (Ad5) and 35 (Ad35). Generally speaking, adenoviral stocks that can be employed as a source of the cited adenovirus are currently available from the American Type Culture Collection (ATCC, Rockville, Md.), or from any other source. Moreover, such adenoviruses have been the subject of numerous publications describing their sequence, organization and biology, allowing the artisan to apply them. Adenoviral vectors, methods of producing adenoviral vectors, and methods of using adenoviral vectors are disclosed, for example in U.S. Pat. No. 6,133,028, U.S. Pat. No. 6,040,174, U.S. Pat. No. 6,110,735, U.S. Pat. No. 6,399,587, WO 00/50573 and EP 1016711 for group C adenoviral vectors and for example in U.S. Pat. No. 6,492,169 and WO 02/40665 for non-group C adenoviral vectors.

In certain embodiments, the adenoviral vector of the present disclosure is replication-competent. The term “replication-competent” as used herein refers to an adenoviral vector capable of replicating in a host cell in the absence of any trans-complementation. In the context of the present disclosure, this term also encompasses replication-selective or conditionally-replicative adenoviral vectors which are engineered to replicate better or selectively in cancer or hyperproliferative host cells. Examples of such replication-competent adenoviral vectors are well known in the art and readily available to those skill in the art (see, for example, Hernandez-Alcoceba et al., 2000, Human Gene Ther. 11, 2009-2024; Nemunaitis et al., 2001, Gene Ther. 8, 746-759; Alemany et al., 2000, Nature Biotechnology 18, 723-727).

Replication-competent adenoviral vectors according to the disclosure can be a wild-type adenovirus genome or can be derived therefrom by introducing modifications into the viral genome, e.g., for the purpose of generating a conditionally-replicative adenoviral vector. Such modification(s) include the deletion, insertion and/or mutation of one or more nucleotide(s) in the coding sequences and/or the regulatory sequences. Typical modifications are those that render said replication-competent adenoviral vector dependent on cellular activities specifically present in a tumor or cancerous cell. In this regard, viral gene(s) that become dispensable in tumor cells, such as the genes responsible for activating the cell cycle through p53 or Rb binding, can be completely or partially deleted or mutated. By way of illustration, such conditionally-replicative adenoviral vectors can be engineered by the complete deletion of the adenoviral MB gene encoding the 55 kDa protein or the complete deletion of the MB region to abrogate p53 binding (see for example U.S. Pat. No. 5,801,029 and U.S. Pat. No. 5,846,945). This prevents the virus from inactivating tumor suppression in normal cells, which means that the virus cannot replicate. However, the virus will replicate and lyse cells that have shut off p53 or Rb expression through oncogenic transformation. As another example, the complete deletion of the E1A region makes the adenoviral vector dependent on intrinsic or IL-6-induced E1A-like activities. Optionally, an inactivating mutation may also be introduced in the E1A region to abrogate binding to the Rb. Rb defective mutation/deletion is typically introduced within the E1A CR1 and/or CR2 domain (see for example WO00/24408). In a second strategy optionally or in combination to the first approach, native viral promoters controlling transcription of the viral genes can be replaced with tissue or tumor-specific promoters. By way of illustration, regulation of the E1A and/or the E1B genes can be placed under the control of a tumor-specific promoter such as the PSA, the kallikrein, the probasin, the AFP, the a-fetoprotein or the telomerase reverse transcriptase (TERT) promoter (see for example U.S. Pat. No. 5,998,205, WO 99/25860, U.S. Pat. No. 5,698,443 and WO 00/46355) or a cell-cycle specific promoter such as E2F-1 promoter (WO00/15820 and WO01/36650). Typical in this context is the exemplary vector designated ONYX-411 which combines a Rb defective deletion of 8 amino acid residues within the MA CR2 domain and the use of E2F-1 promoter to control expression of both the E1A and E4 viral genes.

In certain embodiments, the adenoviral vector of the disclosure is replication-defective. Replication-defective adenoviral vectors are known in the art and can be defined as being deficient in one or more regions of the adenoviral genome that are essential to the viral replication (e.g., E1, E2 or E4 or combination thereof), and thus unable to propagate in the absence of trans-complementation (e.g., provided by either complementing cells or a helper virus). The replication-defective phenotype is obtained by introducing modifications in the viral genome to abrogate the function of one or more viral gene(s) essential to the viral replication. Typical replication-defective vectors are E1-deleted, and thus defective in E1 function. Such E1-deleted adenoviral vectors include those described in U.S. Pat. No. 6,063,622; U.S. Pat. No. 6,093,567; WO 94/28152; WO 98/55639 and EP 974 668 (the disclosures of all of these publications are hereby incorporated herein by reference). A typical E1 deletion covers approximately the nucleotides (nt) 459 to 3328 or 459 to 3510, by reference to the sequence of the human adenovirus type 5 (disclosed in the Genbank under the accession number M 73260 and in Chroboczek et al., 1992, Virol. 186, 280-285).

Furthermore, the adenoviral backbone of the vector may comprise modifications (e.g. deletions, insertions or mutations) in additional viral region(s), to abolish the residual synthesis of the viral antigens and/or to improve long-term expression of the nucleic acid molecules in the transduced cells (see for example WO 94/28152; Lusky et al., 1998, J. Virol 72, 2022-2032; Yeh et al., 1997, FASEB J. 11, 615-623). In this context, the present disclosure contemplates the use of adenoviral vectors lacking E1, or E1 and E2, or E1 and E3, or E1 and E4, or E1 and E2 and E3, or E1 and E2 and E4, or E1 and E3 and E4, or E1 and E2 and E3 and E4 functions. An adenoviral vector defective for E2 function may be deleted of all or part of the E2 region (typically within the E2A or alternatively within the E2B or within both the E2A and the E2B regions) or comprises one or more mutations, such as the thermosensitive mutation of the DBP (DNA Binding Protein) encoding gene (Ensinger et al., J. Virol. 10 (1972), 328-339). The adenoviral vector may also be deleted of all or part of the E4 region (see, for example, EP 974 668 and WO 00/12741). An exemplary E4 deletion covers approximately the nucleotides from position 32994 to position 34998, by reference to the sequence of the human adenovirus type 5. In addition, deletions within the non-essential E3 region (e.g. from Ad5 position 28597 to position 30469) may increase the cloning capacity, but it may be advantageous to retain the E3 sequences coding for gp19k, 14.7K and/or RID allowing to escape the host immune system (Gooding et al., 1990, Critical Review of Immunology 10, 53-71) and inflammatory reactions (EP 00 440 267.3). It is also conceivable to employ a minimal (or gutless) adenoviral vector which lacks all functional genes including early (E1, E2, E3 and E4) and late genes (L1, L2, L3, L4 and L5) with the exception of cis-acting sequences (see for example Kovesdi et al., 1997, Current Opinion in Biotechnology 8, 583-589; Yeh and Perricaudet, 1997, FASEB 11, 615-623; WO 94/12649; and WO 94/28152). The replication-deficient adenoviral vector may be readily engineered by one skilled in the art, taking into consideration the required minimum sequences, and is not limited to these exemplary embodiments.

The nucleic acid molecule of the present disclosure can be inserted in any location of the adenoviral genome, with the exception of the cis-acting sequences. Typically, it is inserted in replacement of a deleted region (E1, E3 and/or E4), with a special preference for the deleted E1 region. In addition, the expression cassette may be positioned in sense or antisense orientation relative to the natural transcriptional direction of the region in question.

A retroviral vector is also suitable in the context of the present disclosure. Retroviruses are a class of integrative viruses which replicate using a virus-encoded reverse transcriptase, to replicate the viral RNA genome into double stranded DNA which is integrated into chromosomal DNA of the infected cells. The numerous vectors described in the literature may be used within the framework of the present disclosure and especially those derived from murine leukemia viruses, especially Moloney (Gilboa et al., 1988, Adv. Exp. Med. Biol. 241, 29) or Friend's FB29 strains (WO 95/01447). Generally, a retroviral vector is deleted of all or part of the viral genes gag, pol and env and retains 5′ and 3′ LTRs and an encapsidation sequence. These elements may be modified to increase expression level or stability of the retroviral vector. Such modifications include the replacement of the retroviral encapsidation sequence by one of a retrotransposon such as VL30 (U.S. Pat. No. 5,747,323). The nucleic acid molecule of the disclosure can be inserted downstream of the encapsidation sequence, typically in opposite direction relative to the retroviral genome.

A poxviral vector is also suitable in the context of the present disclosure. Poxviruses are a group of complex enveloped viruses that distinguish from the above-mentioned viruses by their large DNA genome and their cytoplasmic site of replication. The genome of several members of poxyiridae has been mapped and sequenced. It is a double-stranded DNA of approximately 200 kb coding for about 200 proteins of which approximately 100 are involved in virus assembly. In the context of the present disclosure, a poxyiral vector may be obtained from any member of the poxyiridae, in particular canarypox, fowlpox and vaccinia virus, the latter being typical. Suitable vaccinia viruses include without limitation the Copenhagen strain (Goebel et al., 1990, Virol. 179, 247-266 and 517-563; Johnson et al., 1993, Virol. 196, 381-401), the Wyeth strain and the modified Ankara (MVA) strain (Antoine et al., 1998, Virol. 244, 365-396). The general conditions for constructing poxvirus comprising a nucleic acid molecule are well known in the art (see for example EP 83 286; EP 206 920 for Copenhagen vaccinia viruses and Mayr et al., 1975, Infection 3, 6-14; Sutter and Moss, 1992, Proc. Natl. Acad. Sci. USA 89, 10847-10851, U.S. Pat. No. 6,440,422 for MVA viruses). The nucleic acid molecule of the present disclosure is typically inserted within the poxyiral genome in a non-essential locus, such as non-coding intergenic regions or any gene for which inactivation or deletion does not significantly impair viral growth and replication. Thymidine kinase gene is particularly appropriate for insertion in Copenhagen vaccinia viruses (Hruby et al., 1983, Proc. Natl. Acad. Sci. USA 80, 3411-3415; Weir et al., 1983, J. Virol. 46, 530-537). As far as MVA is concerned, insertion of the nucleic acid molecule can be performed in any of the excisions I to VII, and typically in excision H or III (Meyer et al., 1991, J. Gen. Virol. 72, 1031-1038; Sutter et al., 1994, Vaccine 12, 1032-1040) or in D4R locus. For fowlpox virus, although insertion within the thymidine kinase gene may be considered, the nucleic acid molecule is typically introduced into a non-coding intergenic region (see for example EP 314 569 and U.S. Pat. No. 5,180,675). One may also envisage insertion in an essential viral locus provided that the defective function be supplied in trans, via a helper virus or by expression in the producer cell line. Suitable poxyiral vectors can be readily generated from wild type poxviruses available in recognized collections such as ATCC (fowlpox ATCC VR-251, monkey pox ATCC VR-267, swine pox ATCC VR-363, canarypox ATCC VR-111, cowpox ATCC VR-302) or ICTV (Canberra, Australia) (Copenhagen virus code 58.1.1.0.001; GenBank accession number M35027).

In certain embodiments, the vectors of the disclosure comprise the nucleic acid molecule of the disclosure in a form suitable for its expression in a host cell or organism, which means that the nucleic acid molecule is placed under the control of one or more regulatory sequences, selected on the basis of the vector type and/or host cell, which is operatively linked to the nucleic acid molecule to be expressed. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability). “Operably linked” is intended to mean that the nucleic acid molecule of interest is linked to the regulatory sequence(s) in a manner which allows for expression of the nucleic acid molecule (e.g., in a host cell or organism). It will be appreciated by those skilled in the art that the design of the expression vector can depend on such factors as the choice of the host cell to be transformed, the level of expression of protein desired, etc.

Regulatory sequences include promoters which direct constitutive expression of a nucleic acid molecule in many types of host cell and those which direct expression of the nucleotide sequence only in certain host cells (e.g., tissue-specific regulatory sequences) or in response to specific events or exogenous factors (e.g. by temperature, nutrient additive, hormone or other ligand).

Suitable regulatory sequences useful in the context of the present disclosure include, but are not limited to, the left promoter from bacteriophage lambda, the lac, TRP, and TAC promoters from E. coli, the early and late promoters from SV40, the cytomegalovirus (CMV) immediate early promoter or enhancer (Boshart et al., 1985, Cell 41, 521-530), the adenovirus early and late promoters, the phosphoglycero kinase (PGK) promoter (Hitzeman et al., 1983, Science 219, 620-625; Adra et al., 1987, Gene 60, 65-74), the thymidine kinase (TK) promoter of herpes simplex virus (HSV)-1 and retroviral long-terminal repeats (e.g. MoMuLV and Rous sarcoma virus (RSV) LTRs). Suitable promoters useful to drive expression of the nucleic acid molecule of the disclosure in a poxyiral vector include the 7.5K, H5R, TK, p28, p11 or K1L promoters of vaccinia virus. Alternatively, one may use a synthetic promoter such as those described in Chakrabarti et al. (1997, Biotechniques 23, 1094-1097), Hammond et al. (1997, J. Virological Methods 66, 135-138) and Kumar and Boyle (1990, Virology 179, 151-158) as well as chimeric promoters between early and late poxyiral promoters.

Inducible promoters are regulated by exogenously supplied compounds, and include, without limitation, the zinc-inducible metallothionein (MT) promoter (Mc Ivor et al., 1987, Mol. Cell. Biol. 7, 838-848), the dexamethasone (Dex)-inducible mouse mammary tumor virus (MMTV) promoter, the T7 polymerase promoter system (WO 98/10088), the ecdysone insect promoter (No et al., 1996, Proc. Natl. Acad. Sci. USA 93, 3346-3351), the tetracycline-repressible promoter (Gossen et al., 1992, Proc. Natl. Acad. Sci. USA 89, 5547-5551), the tetracycline-inducible promoter (Kim et al., 1995, J. Virol. 69, 2565-2573), the RU486-inducible promoter (Wang et al., 1997, Nat. Biotech. 15, 239-243 and Wang et al., 1997, Gene Ther. 4, 432-441) and the rapamycin-inducible promoter (Magari et al., 1997, J. Clin. Invest. 100, 2865-2872).

The regulatory sequences in use in the context of the present disclosure can also be tissue-specific to drive expression of the nucleic acid molecule in the tissues where therapeutic benefit is desired. Exemplary liver-specific regulatory sequences include but are not limited to those of HMG-CoA reductase (Luskey, 1987, Mol. Cell. Biol. 7, 1881-1893); sterol regulatory element 1 (SRE-1; Smith et al., 1990, J. Biol. Chem. 265, 2306-2310); albumin (Pinkert et al., 1987, Genes Dev. 1, 268-277); phosphoenol pyruvate carboxy kinase (PEPCK) (Eisenberger et al., 1992, Mol. Cell. Biol. 12, 1396-1403); human C-reactive protein (CRP) (Li et al., 1990, J. Biol. Chem. 265, 4136-4142); human glucokinase (Tanizawa et al., 1992, Mol. Endocrinology. 6, 1070-1081); cholesterol 7-alpha hydroylase (CYP-7) (Lee et al., 1994, J. Biol. Chem. 269, 14681-14689); alpha-1 antitrypsin (Ciliberto et al., 1985, Cell 41, 531-540); insulin-like growth factor binding protein (IGFBP-1) (Babajko et al., 1993, Biochem Biophys. Res. Comm. 196, 480-486); human transferrin (Mendelzon et al., 1990, Nucl. Acids Res. 18, 5717-5721); collagen type I (Houglum et al., 1994, J. Clin. Invest. 94, 808-814) and FIX (U.S. Pat. No. 5,814,716) genes. Exemplary prostate-specific regulatory sequences include but are not limited to those of the prostatic acid phosphatase (PAP) (Balms et al., 1994, Biochim. Biophys. Acta. 1217, 188-194); prostatic secretory protein 94 (PSP 94) (Nolet et al., 1991, Biochim. Biophys. Acta 1089, 247-249); prostate specific antigen complex (Kasper et al., 1993, J. Steroid Biochem. Mol. Biol. 47, 127-135); human glandular kallikrein (hgt-1) (Lilja et al., 1993, World J. Urology 11, 188-191) genes. Exemplary pancreas-specific regulatory sequences include but are not limited to those of pancreatitis associated protein promoter (Dusetti et al., 1993, J. Biol. Chem. 268, 14470-14475); elastase 1 transcriptional enhancer (Kruse et al., 1993, Genes and Development 7, 774-786); pancreas specific amylase and elastase enhancer/promoter (Wu et al., 1991, Mol. Cell. Biol. 11, 4423-4430; Keller et al., 1990, Genes & Dev. 4, 1316-1321); pancreatic cholesterol esterase gene promoter (Fontaine et al., 1991, Biochemistry 30, 7008-7014) and the insulin gene promoter (Edlund et al., 1985, Science 230, 912-916). Exemplary neuron-specific regulatory sequences include but are not limited to neuron-specific enolase (NSE) (Forss-Petter et al., 1990, Neuron 5, 187-197) and the neurofilament (Byrne and Ruddle, 1989, Proc. Natl. Acad. Sci. USA 86, 5473-5477) gene promoters. Exemplary regulatory sequences for expression in the brain include but are not limited to the neurofilament heavy chain (NF-H) promoter (Schwartz et al., 1994, J. Biol. Chem. 269, 13444-13450). Exemplary lymphoid-specific regulatory sequences include but are not limited to the human CGL1/granzyme B promoter (Hanson et al., 1991, J. Biol. Chem. 266, 24433-24438); terminal deoxy transferase (TdT), lymphocyte specific tyrosine protein kinase (p561ck) promoters (Lo et al., 1991, Mol. Cell. Biol. 11, 5229-5243); the human CD2 promoter/enhancer (Lake et al., 1990, EMBO J. 9, 3129-3136), the human NK and T cell specific activation (NKG5) (Houchins et al., 1993, Immunogenetics 37, 102-107), T cell receptor (Winoto and Baltimore, 1989, EMBO J. 8, 729-733) and immunoglobulin (Banerji et al., 1983, Cell 33, 729-740; Queen and Baltimore, 1983, Cell 33, 741-748) promoters. Exemplary colon-specific regulatory sequences include but are not limited to pp 60c-src tyrosine kinase (Talamonti et al., 1993, J. Clin. Invest 91, 53-60); organ-specific neoantigens (OSNs), mw 40 kDa (p40) (Ilantzis et al., 1993, Microbiol. Immunol. 37, 119-128); and colon specific antigen-P promoter (Sharkey et al., 1994, Cancer 73, 864-877) promoters. Exemplary regulatory sequences for expression in mammary gland and breast cells include but are not limited to the human alpha-lactalbumin (Thean et al., 1990, British J. Cancer. 61, 773-775) and milk whey (U.S. Pat. No. 4,873,316) promoters. Exemplary muscle-specific regulatory sequences include but are not limited to SM22 (WO 98/15575; WO 97/35974), the desmin (WO 96/26284), mitochondrial creatine kinase (MCK) promoters, and the chimeric promoter disclosed in EP 1310561. Exemplary lung-specific regulatory sequences include but are not limited to the CFTR and surfactant promoters.

Additional promoters suitable for use in this disclosure can be taken from genes that are preferentially expressed in proliferative tumor cells. Such genes can be identified for example by display and comparative genomic hybridization (see for example U.S. Pat. Nos. 5,759,776 and 5,776,683). Exemplary tumor specific promoters include but are not limited to the promoters of the MUC-1 gene overexpressed in breast and prostate cancers (Chen et al., 1995, J. Clin. Invest. 96, 2775-2782), of the Carcinoma Embryonic Antigen (CEA)-encoding gene overexpressed in colon cancers (Schrewe et al., 1990, Mol. Cell. Biol. 10, 2738-2748), of the ERB-2 encoding gene overexpressed in breast and pancreas cancers (Harris et al., 1994, Gene Therapy 1, 170-175), of the alpha-foetoprotein gene overexpressed in liver cancers (Kanai et al., 1997, Cancer Res. 57, 461-465), of the telomerase reverse transcriptase (TERT) (WO99/27113, WO 02/053760 and Horikawa et al., 1999, Cancer Res. 59, 826), hypoxia-responsive element (HRE), autocrine motility factor receptor, L plasmin and hexokinase II.

Those skilled in the art will appreciate that the regulatory elements controlling the expression of the nucleic acid molecule of the disclosure may further comprise additional elements for proper initiation, regulation and/or termination of transcription and translation into the host cell or organism. Such additional elements include but are not limited to non-coding exon/intron sequences, transport sequences, secretion signal sequences, nuclear localization signal sequences, IRES, polyA transcription termination sequences, tripartite leader sequences, sequences involved in replication or integration. Illustrative examples of introns suitable in the context of the disclosure include those isolated from the genes encoding alpha or beta globin (i.e. the second intron of the rabbit beta globin gene; Green et al., 1988, Nucleic Acids Res. 16, 369; Karasuyama et al., 1988, Eur. J. Immunol. 18, 97-104), ovalbumin, apolipoprotein, immunoglobulin, factor IX, and factor VIII, the SV40 16S/19S intron (Okayma and Berg, 1983, Mol. Cell. Biol. 3, 280-289) as well as synthetic introns such as the intron present in the pCI vector (Promega Corp, pCI mammalian expression vector E1731) made of the human beta globin donor fused to the mouse immunoglobin. Where secretion of the fusion protein is desired, appropriate secretion signals are incorporated into the vector. The signal sequence can be endogenous to the fusion protein or heterologous to both entities involved in the fusion protein. The person of ordinary skill in the art would be aware of the numerous regulatory sequences that are useful in expression vectors.

In addition, the vector of the disclosure can further comprise one or more transgenes (i.e. a gene of interest to be expressed together with the nucleic acid molecule of the disclosure in a host cell or organism). Desirably, the expression of the transgene has a therapeutic or protective activity to the disease or illness condition for which the vector of the present disclosure is being given. Suitable transgenes include without limitation genes encoding (i) tumor proliferation inhibitors and/or (ii) at least one specific antigen against which an immune response is desired. In a typical form of the present disclosure, the transgene product and the fusion protein act synergistically in the induction of immune responses or in providing a therapeutic (e.g. antitumoral) benefit. Accordingly, such combinations are not only suitable for immunoprophylaxis of diseases, but surprisingly also for immunotherapy of diseases such as viral, bacterial or parasitic infections, and also chronic disorders such as cancers.

Tumor proliferation inhibitors act by directly inhibiting cell growth, or killing the tumor cells. Representative examples of tumor proliferation inhibitors include toxins and suicide genes. Representative examples of toxins include without limitation ricin (Lamb et al., 1985, Eur. J. Biochem. 148, 265-270), diphtheria toxin (Tweten et al., 1985, J. Biol. Chem. 260, 10392-10394), cholera toxin (Mekalanos et al., 1983, Nature 306, 551-557; Sanchez and Holmgren, 1989, Proc. Natl. Acad. Sci. USA 86, 481-485), gelonin (Stirpe et al., 1980, J. Biol. Chem. 255, 6947-6953), antiviral protein (Barbieri et al., 1982, Biochem. J. 203, 55-59; Irvin et al., 1980, Arch. Biochem. Biophys. 200, 418-425), tritin, Shigella toxin (Calderwood et al., 1987, Proc. Natl. Acad. Sci. USA 84, 4364-4368; Jackson et al., 1987, Microb. Path. 2, 147-153) and Pseudomonas exotoxin A (Carroll and Collier, 1987, J. Biol. Chem. 262, 8707-8711).

Specific antigens are typically those susceptible to confer an immune response, specific and/or nonspecific, antibody and/or cell-mediated, against a given pathogen (virus, bacterium, fungus or parasite) or against a non-self antigen (e.g. a tumor-associated antigen). Typically, the selected antigen comprises an epitope that binds to, and is presented onto the cell surface by MHC class I proteins. Representative examples of specific antigens include without limitation: antigen(s) of the Hepatitis B surface antigen are well known in the art and include, inter alia, those PreS1, Pars2 S antigens set forth described in European Patent applications EP 414 374; EP 304 578, and EP 198 474. Antigens of the Hepatitis C virus including any immunogenic antigen or fragment thereof selected from the group consisting of the Core (C), the envelope glycoprotein E1, E2, the non-structural polypeptide NS2, NS3, NS4 (NS4a and/or NS4b), NS5 (NS5a and/or NS5b) or any combination thereof (e.g. NS3 and NS4, NS3 and NS4 and NS5b) Antigen(s) of the HIV-1 virus, especially gp120 and gp160 (as described WO 87/06260). Antigen(s) derived from the Human Papilloma Virus (HPV) considered to be associated with genital warts (HPV 6 or HPV 11 and others), and cervical cancer (HPV16, HPV18, HPV 31, HPV-33 and others). Contemplated HPV antigens are selected among the group consisting of E5, E6, E7, L1, and L2 either individually or in combination (see for example WO 94/00152, WO 94/20137, WO 93/02184, WO 90/10459, and WO 92/16636). Contemplated in the context of the disclosure are membrane anchored forms of non-oncogenic variants of the early HPV-16 E6 and/or E7 antigens (as described in WO 99/03885) that are particularly suitable to achieve an anti-tumoral effect against an HPV-associated cancer. Antigens from parasites that cause malaria. For example, typical antigens from Plasmodia falciparum include RTS (WO 93/10152), and TRAP (WO 90/01496). Other plasmodia antigens that are likely candidates are P. falciparum. MSP1, AMA1, MSP3, EBA, GLURP, RAPT, RAP2, Sequestrin, PfEMP1, Pf332, LSA1, LSA3, STARP, SALSA, PfEXP1, Pfs25, Pfs28, PFS27125, Pfs16, Pfs48/45, Pfs230 and their analogues in other Plasmodium species.

Other suitable antigens include tumour-associated antigens such as those associated with prostrate, breast, colorectal, lung, pancreatic, renal, liver, bladder, sarcoma or melanoma cancers. Exemplary antigens include MAGE 1, 3 and MAGE 4 or other MAGE antigens (WO 99/40188), PRAME, BAGE, Lage (also known as NY Eos 1) SAGE and HAGE (WO 99/53061) or GAGE (Robbins and Kawakami, 1996. Current Opinions in Immunol. 8, pps 628-636). Other suitable tumor-associated antigens include those known as prostase, including Prostate specific antigen (PSA), PAP, PSCA, PSMA. Prostase nucleotide sequence and deduced polypeptide sequence and homologs are disclosed in Ferguson, et al. (1999, Proc. Natl. Acad. Sci. USA. 96, 3114-3119) and WO 98/12302 WO 98/20117 and WO 00/04149. Other suitable tumour-associated antigens include those associated with breast cancer, such as BRCA-1, BRCA-2 and MUC-1 (see for example WO 92/07000).

The transgene in use in the present disclosure is placed under the control of appropriate regulatory elements to permit its expression in the selected host cell or organism in either a constitutive or inducible fashion. The choice of such regulatory elements is within the reach of the skilled artisan. It is typically selected from the group consisting of constitutive, inducible, tumor-specific and tissue-specific promoters as described above in connection with the expression of the fusion protein of the present disclosure. In one example, the transgene is placed under control of the CMV promoter to ensure high level expression.

The transgene in use in the present disclosure can be inserted in any location of the vector. According to one alternative, it is placed typically not in close proximity of the nucleic acid molecule of the disclosure. According to another alternative it can be placed in antisense orientation with respect to the nucleic acid molecule, in order to avoid transcriptional interference between the two expression cassettes. For example, in an adenoviral genome, the transgene can be inserted in a different deleted region with respect to the nucleic acid molecule of the disclosure (E1, E3 and/or E4) or in the same deleted region as said nucleic acid molecule but in antisense orientation to one another.

Introducing the nucleic acid molecule of the disclosure into a vector backbone can proceed by any genetic engineering strategy appropriate in the art for any kind of vectors such as by methods described in Sambrook et al. (2001, Molecular Cloning—A Laboratory Manual, Cold Spring Harbor Laboratory). Typically, for the introduction of the nucleic acid molecule into an adenoviral vector, a bacterial plasmid comprising the fusion-encoding nucleic acid molecule is engineered to replace an adenoviral gene required for replication or assembly (e.g. E1) with the substitute nucleic acid molecule. The plasmid is then used as a shuttle vector, and combined with a second plasmid containing the complementary portion of the adenovirus genome, permitting homologous recombination to occur by virtue of overlapping adenovirus sequences in the two plasmids. The recombination can be done directly in a suitable mammalian host (such as 293 as described in Graham and Prevect, 1991, Methods in Molecular Biology, Vol 7 “Gene Transfer and Expression Protocols”; Ed E. J. Murray, The Human Press Inc, Clinton, N.J.), or else in yeast YAC clones or E. coli (as described in WO 96/17070). The completed adenovirus genome is subsequently transfected into mammalian host cells for replication and viral encapsidation.

The present disclosure also encompasses vectors of the disclosure or particles thereof that have been modified to allow preferential targeting of a particular target cell. A characteristic feature of targeted vectors/particles of the disclosure (of both viral and non-viral origins, such as polymer- and lipid-complexed vectors) is the presence at their surface of a targeting moiety capable of recognizing and binding to a cellular and surface-exposed component. Such targeting moieties include without limitation chemical conjugates, lipids, glycolipids, hormones, sugars, polymers (e.g. PEG, polylysine, PEI and the like), peptides, polypeptides (for example JTS1 as described in WO 94/40958), oligonucleotides, vitamins, antigens, lectins, antibodies and fragments thereof. They are typically capable of recognizing and binding to cell-specific markers, tissue-specific markers, cellular receptors, viral antigens, antigenic epitopes or tumor-associated markers. In this regard, cell targeting of adenoviruses can be carried out by genetic modification of the viral gene encoding the capsid polypeptide present on the surface of the virus (e.g. fiber, penton and/or pIX). Examples of such modifications are described in literature (for example in Wickam et al., 1997, J. Viral. 71, 8221-8229; Amberg et al., 1997, Virol. 227, 239-244; Michael et al., 1995, Gene Therapy 2, 660-668; WO 94/10323, EP 02 360204 and WO 02/96939). To illustrate, inserting a sequence coding for EGF within the sequence encoding the adenoviral fiber will allow to target EGF receptor expressing cells. The modification of poxyiral tropism can also be achieved as described in EP 1 146 125. Other methods for cell specific targeting can be achieved by the chemical conjugation of targeting moieties at the surface of a viral particle.

In certain embodiments, the present disclosure relates to infectious viral particles comprising the above-described nucleic acid molecules or vectors of the present disclosure.

The disclosure also relates to a process for producing an infectious viral particle, comprising the steps of: (a) introducing the viral vector of the disclosure into a suitable cell line, (b) culturing said cell line under suitable conditions so as to allow the production of said infectious viral particle, and (c) recovering the produced infectious viral particle from the culture of said cell line, and (d) optionally purifying said recovered infectious viral particle.

The vector containing the nucleic acid molecule of the disclosure can be introduced into an appropriate cell line for propagation or expression using well-known techniques readily available to the person of ordinary skill in the art. These include, but are not limited to, microinjection of minute amounts of DNA into the nucleus of a cell (Capechi et al., 1980, Cell 22, 479-488), CaPO.sub.4-mediated transfection (Chen and Okayama, 1987, Mol. Cell Biol. 7, 2745-2752), DEAE-dextran-mediated transfection, electroporation (Chu et al., 1987, Nucleic Acid Res. 15, 1311-1326), lipofection/liposome fusion (Feigner et al., 1987, Proc. Natl. Acad. Sci. USA 84, 7413-7417), particle bombardment (Yang et al., 1990, Proc. Natl. Acad. Sci. USA 87, 9568-9572), gene guns, transduction, infection (e.g. with an infective viral particle), and other techniques such as those found in Sambrook, et al. (Molecular Cloning: A Laboratory Manual, Cold Spring Harbor Laboratory, Cold Spring Harbor Laboratory Press, Cold Spring Harbor, N.Y., 2001).

When the vector of the disclosure is defective, the infectious particles are usually produced in a complementation cell line or via the use of a helper virus, which supplies in trans the non-functional viral genes. For example, suitable cell lines for complementing adenoviral vectors include the 293 cells (Graham et al., 1997, J. Gen. Virol. 36, 59-72) as well as the PER-C6 cells (Fallaux et al., 1998, Human Gene Ther. 9, 1909-1917) commonly used to complement the E1 function. Other cell lines have been engineered to complement doubly defective adenoviral vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; WO94/28152 and WO97/04119). The infectious viral particles may be recovered from the culture supernatant but also from the cells after lysis and optionally are further purified according to standard techniques (chromatography, ultracentrifugation in a cesium chloride gradient as described for example in WO 96/27677, WO 98/00524, WO 98/22588, WO 98/26048, WO 00/40702, EP 1016700 and WO 00/50573).

The disclosure also relates to host cells which comprise the nucleic acid molecules, vectors or infectious viral particles of the disclosure described herein. For the purpose of the disclosure, the term “host cell” should be understood broadly without any limitation concerning particular organization in tissue, organ, or isolated cells. Such cells may be of a unique type of cells or a group of different types of cells and encompass cultured cell lines, primary cells and proliferative cells.

Host cells therefore include prokaryotic cells, lower eukaryotic cells such as yeast, and other eukaryotic cells such as insect cells, plant and higher eukaryotic cells, such as vertebrate cells and, with a special preference, mammalian (e.g. human or non-human) cells. Suitable mammalian cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g. skeletal muscle, cardiac muscle or smooth muscle) or fibroblasts. Typical host cells include Escherichia coli, Bacillus, Listeria, Saccharomyces, BHK (baby hamster kidney) cells, MDCK cells (Madin-Darby canine kidney cell line), CRFK cells (Crandell feline kidney cell line), CV-1 cells (African monkey kidney cell line), COS (e.g., COS-7) cells, chinese hamster ovary (CHO) cells, mouse NIH/3T3 cells, HeLa cells and Vero cells. Host cells also encompass complementing cells capable of complementing at least one defective function of a replication-defective vector of the disclosure (e.g. adenoviral vector) such as those cited above.

The host cell of the disclosure can contain more than one nucleic acid molecule, vector or infectious viral particle of the disclosure. Further it can additionally comprise a vector encoding a transgene, e.g. a transgene as described above. When more than one nucleic acid molecule, vector or infectious viral particle is introduced into a cell, the nucleic acid molecules, vectors or infectious viral particles can be introduced independently or co-introduced.

Moreover, according to a specific embodiment, the host cell of the disclosure can be further encapsulated. Cell encapsulation technology has been previously described (Tresco et al., 1992, ASAJO J. 38, 17-23; Aebischer et al., 1996, Human Gene Ther. 7, 851-860). According to said specific embodiment, transfected or infected eukaryotic host cells are encapsulated with compounds which form a microporous membrane and said encapsulated cells can further be implanted in vivo. Capsules containing the cells of interest may be prepared employing hollow microporous membranes (e.g. Akzo Nobel Faser AG, Wuppertal, Germany; Deglon et al. 1996, Human Gene Ther. 7, 2135-2146) having a molecular weight cutoff appropriate to permit the free passage of proteins and nutrients between the capsule interior and exterior, while preventing the contact of transplanted cells with host cells.

Still a further aspect of the present disclosure is a method for recombinantly producing the fusion protein, employing the vectors, infectious viral particles and/or host cells of the disclosure. The method for producing the fusion protein comprises introducing a vector or an infectious viral particle of the disclosure into a suitable host cell to produce a transfected or infected host cell, culturing in-vitro said transfected or infected host cell under conditions suitable for growth of the host cell, and thereafter recovering said fusion protein from said culture, and optionally, purifying said recovered fusion protein. It is expected that those skilled in the art are knowledgeable in the numerous expression systems available for expression of the fusion proteins of the disclosure in appropriate host cells.

The host cell of the disclosure is typically produced by transfecting/infecting a host cell with one or more recombinant molecules, (e.g. a vector of the disclosure) comprising one or more nucleic acid molecules of the present disclosure. Recombinant DNA technologies can be used to improve expression of the nucleic acid molecule in the host cell by manipulating, for example, the number of copies of the nucleic acid molecule within a host cell, the efficiency with which the nucleic acid molecule is transcribed, the efficiency with which the resultant transcripts are translated, the efficiency of post-translational modifications and the use of appropriate selection. Recombinant techniques useful for increasing the expression of nucleic acid molecules of the present disclosure include, but are not limited to, the use of high-copy number vectors, addition of vector stability sequences, substitution or modification of one or more transcriptional regulatory sequences (e.g., promoters, operators, enhancers), substitution or modification of translational regulatory sequences (e.g., ribosome binding sites, Shine-Dalgamo sequences), modification of nucleic acid molecule of the present disclosure to correspond to the codon usage of the host cell, and deletion of sequences that destabilize transcripts.

Host cells of the present disclosure can be cultured in conventional fermentation bioreactors, flasks, and petri plates. Culturing can be carried out at a temperature, pH and oxygen content appropriate for a given host cell. No attempts to describe in detail the various methods known for the expression of proteins in prokaryote and eukaryote cells will be made here. In one embodiment, the vector is a plasmid carrying the fusion-encoding nucleic acid molecule in operative association with appropriate regulatory elements. Typical host cells in use in the method of the disclosure are mammalian cell lines, yeast cells and bacterial cells.

Where the fusion protein is not secreted outside the producing cell or where it is not secreted completely, it can be recovered from the cell by standard disruption procedures, including freeze thaw, sonication, mechanical disruption, use of lysing agents and the like. If secreted, it can be recovered directly from the culture medium. The fusion protein can then be recovered and purified by well-known purification methods including ammonium sulfate precipitation, acid extraction, gel electrophoresis, reverse phase chromatography, size exclusion chromatography, ion exchange chromatography, affinity chromatography, phosphocellulose chromatography, hydrophobic-interaction chromatography, hydroxylapatite chromatography, lectin chromatography, or high performance liquid chromatography. The conditions and technology used to purify a particular fusion protein of the disclosure will depend on the synthesis method and on factors such as net charge, molecular weight, hydrophobicity, hydrophilicity and will be apparent to those having skill in the art. It is also understood that depending upon the host cell used for the recombinant production of the fusion proteins described herein, the fusion proteins can have various glycosylation patterns, or may be non-glycosylated (e.g. when produced in bacteria). In addition, the fusion protein may include an initial methionine in some cases as a result of a host-mediated process.

The fusion protein of the disclosure can be “purified” to the extent that it is substantially free of cellular material. The level of purification will be based on the intended use. The critical feature is that the preparation allows for the desired function of the fusion protein, even if in the presence of considerable amounts of other components. In some uses, “substantially free of cellular material” includes preparations of the fusion protein having less than about 30% (by dry weight) other proteins (i.e., contaminating proteins), typically less than about 20% other proteins, more typically less than about 10% other proteins, or even more typically less than about 5% other proteins. When the fusion protein is recombinantly produced, it can also be substantially free of culture medium, i.e., culture medium represents less than about 20% of the volume of the protein preparation.

### TERMS

As used herein, the term “conjugate” refers to molecular entities joined by covalent bonds or other arrangement that provides substantially irreversible binding under physiological conditions. For example, two proteins may be conjugated together by a linker polymer, e.g., polypeptide sequence, ethylene glycol polymer. Two proteins may be conjugated together by linking one protein to a ligand and linking the second protein to a receptor, e.g., streptavidin and biotin or an antibody and an epitope.

As used herein, the term “combination with” when used to describe administration with an additional treatment means that the agent may be administered prior to, together with, or after the additional treatment, or a combination thereof.

As used herein, “subject” refers to any animal, typically a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, “amino acid sequence” refers to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include non-naturally occurring amino acids, post-translational modifications of the deduced amino acid sequences, such as amino acid deletions, additions, and modifications such as glycolsylations and addition of lipid moieties.

The term “a nucleic acid sequence encoding” a specified polypeptide refers to a nucleic acid sequence comprising the coding region of a gene or in other words the nucleic acid sequence which encodes a gene product. The coding region may be present in a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

A “virus-like particle” refers to a particle comprising virion proteins but is substantially free of viral genetic material, e.g., viral RNA. Virus-like particles may contain viral proteins from different viruses. See e.g., Guo et al., Enhancement of mucosal immune responses by chimeric influenza HA/SHIV virus-like particles, Virology, 2003, 313 (2):502-13. Virus-like particles may contain lipid membranes and may be constructed to express a variety of antigens on their particle surface ether by expression in viral vectors use to create the particles or by mixing the virus-like particle with an antigen or other polypeptide conjugated to a glycosylphosphatidyl-inositol anchor. See e.g. Skountzou et al., J. Virol. 81 (3):1083-94; Derdak et al., PNAS, 2006, 103 (35) 13144-13149; Poloso et al., Molecular Immunology, 2001, 38:803-816.

As used herein, the article “a” or “an” is intended to refer to one or more unless the context suggests otherwise.

Examples provided herein are not intended to be limiting, and all references cited herein are incorporated by reference in their entirety.

### EXPERIMENTAL

### Cloning and Expression of GIFT9

Mouse GIFT9 cDNA was synthesized at GenScript USA Inc. (Piscataway, N.J.) by aligning mouse GMCSF and IL9 cDNA. The GIFT9 cDNA was then cloned in a bicistronic retrovector AP-2 allowing the expression of GIFT9 and green fluorescent protein. Infectious retroparticles were generated through transfection of 293-GP2 packaging cells (Clontech, Mountain View, Calif.) using PolyFect (Qiagen, Valencia, Calif.). Retroparticles were then used to transduce 293T cells. 293T cells were expanded, and the supernatant was collected and concentrated using Amicon centrifugation columns (Millipore, Billerica, Mass.). GIFT9 expression levels were quantified using mouse GMCSF ELISA kit (eBioscience, San Diego, Calif.).

### Cell Culture

JawsII cells were purchased from ATCC (Manassas, Va.) and cultured in Alpha minimum essential medium (α-MEM) (Thermo Scientific, Waitham, Mass.) supplemented with 10% fetal bovine serum (Wisent Bioproducts, St. Bruno, Canada), 1% penicillin-streptomycin (Thermo Scientific), 1 mM sodium pyruvate (Thermo Scientific) and 5 ng/ml murine GMCSF (R & D systems, Minneapolis, Minn.) in a 5% CO₂ incubator. MC/9 cells were purchased from ATCC and cultured in Dulbecco modified Eagle medium (DMEM) (Thermo Scientific) supplemented with 10% fetal bovine serum, 1% penicillin-streptomycin, 2 mM L-glutamine (Thermo Scientific), 0.05 mM 2-mercaptoethanol (Sigma, St. Louis, Mo.) and 10% Rat T-STIM (BD Biosciences, Franklin Lakes, N.J.) in a 5% CO₂ incubator. 293T cells were purchased from ATCC and cultured in DMEM supplemented with 10% fetal bovine serum, 1% Penicillin-streptomycin in a 5% CO₂ incubator. 293-GP2 cells were purchased from Clontech and cultured in DMEM supplemented with 10% fetal bovine serum, 1% Penicillin-streptomycin in a 5% CO₂ incubator. Recombinant mouse GMCSF and IL9 were purchased from R&D systems.

### Cell Treatment and Western Blot

For cytokine stimulation assay in cell lines, cells were seeded at 9×10⁶/ml in 96-well plate, each well has 100 ml volume. Cells were starved in fetal bovine serum free medium for 5 hours with/without JAK inhibitors, and then were stimulated with 200 nM of GM-CSF, IL-9, both cytokines, and GIFT9 for 15 min at 37° C. Cells were collected, wash once with ice-cold PBS, and lysed with lysis buffer supplemented with protease inhibitors and phosphatase inhibitor. Cell lysates were separated by SDS-PAGE, and Western blot analysis was performed with α-phosphorylated STAT1, STAT3, STAT5 antibodies (Cell signaling, Danvers, Mass.), total STAT1, STAT3, and STAT5 antibodies (cell Signaling) were used as a loading control. A representative result was shown from three independent experiments performed for each panel, the amount of STAT1 phosphorylation signal detected was quantified using the NIH Image J program and normalized against GMCSF+IL9 group. For MβCD treatment, 10 mg/ml MβCD (Sigma) was added to cells 1 hour before cytokine stimulation to disrupt lipid raft formation. JAK1, JAK2, and JAK3 inhibitors were purchased from Selleck Chemicals (Houston, Tex.) dissolved in DMSO, further diluted to desired concentration in PBS, and added to medium 5 hours before cytokine stimulation.

### Co-Immunoprecipitation

For co-immunoprecipitation of GMCSF-Rβ by common γc receptor, MC/9 cells were stimulated with 1 nM of GM-CSF, IL-9, both cytokines, and GIFT9 for 15 min at 37° C. After cytokine stimulation, MC/9 cells were washed twice with PBS and crosslinked with 0.5 mM DSP (Thermo Scientific) for 30 minutes at room temperature. The crosslinking was quenched by 25 mM Tris-HCl, pH 7.4 for 10 minutes. Cells were then washed again with ice-cold PBS and lysed with IP buffer (25 mM Tris-HCl, pH 7.4, 150 mM NaCl, 2 mM EDTA, 0.1% SDS, and 1% NP-40) by incubating on ice for 1 hour. Cell lysates were homogenized by passing through a 27-gauge needle at least 10 times. After removing cell debris by high-speed centrifugation, 1 mg protein from each sample was incubated with 2 mg of α-common γc antibody (Santa Cruz Biotechnology, Santa Cruz, Calif.) at 4° C. overnight. Next day sample-antibody complexes were incubated with 20 ml protein A agarose (Thermo Scientific) at 4° C. for 1.5 hour. Agarose were washed three times with IP buffer, reverse crosslink by incubating with 100 mM DTT in IP buffer at 37° C. for 1.5 hour. Samples were then boiled and separated by SDS-PAGE.

### Immunofluorescence Staining and Confocal Microscopy

MC/9 cells were stimulated with 1 nM of GM-CSF, IL-9, both cytokines, and GIFT9 for 15 min at 37° C. After cytokine stimulation, cells were spin to coverslip, fixed with 3.7% paraformaldehyde, and stained with α-GMCSF-Rβ (R&D systems) and α-common γc (Santa Cruz) antibodies at 4° C. overnight. Next day cells were probed with Donkey anti-rabbit Alexa 488 and Donkey anti-goat Alexa 555 (Invitrogen, Grand Island, N.Y.) at room temperature for 2 hours. Coverslips were then mounted on slides with ProLong Gold Antifade Reagent with DAPI (Invitrogen). The images were captured using a Zeiss LSM 510 Confocal microscope at the integrated Cellular Imaging core of the Winship Cancer Institute at Emory University. For percentages of color pixel colocalization, 10 random selected cells from each group were analyzed by the ZEN software and graphed. P-value was calculated by student t-test.

### BMMC Generation and MTT Assay

BMMCs were generated essentially as previously described [20]. BMMCs were derived from 8-week-old C57BL/6J mice (The Jackson Laboratory, Bar Harbor, Me.). BMMCs were generated by culturing unfractionated C57BL/6 bone marrow cells with 5 ng/ml IL-3 and 50 ng/ml Stem Cell Factor (R&D systems) for 3 weeks. Mast cell phenotype was confirmed by flow cytometry analysis with α-c-kit (BD Biosciences) and α-FcεRIα (Biolegend, San Diego, Calif.) antibodies. BMMC cultures were greater than 97% mast cells at the time of use. MTT assay was performed essentially as described in Wang et al. J Biol Chem, 2012, 287: 25941-25953. BMMCs were cultured with different cytokines for 5 days before analysis.

What we claim:

1. A conjugate comprising a GM-CSF polypeptide and an IL-9 polypeptide.

2. The conjugate of claim 1, wherein GM-CSF and IL-9 polypeptides are connected by a linker.

3. The conjugate of claim 2, wherein the linker is a polypeptide.

4. The conjugate of claim 1, further conjugated to one or more of the group selected from an adjuvant, a cytokine, a co-stimulatory molecule, an antigen, a protein, saccharide, polysaccharide, and a glycoprotein.

5. A pharmaceutical composition comprising a conjugate of claim 1 and a pharmaceutically acceptable excipient.

6. A vaccine comprising a conjugate of claim 1 and an antigen.

7. A method of enhancing an immune response comprising administering a conjugate of claim 1 to a subject in need thereof.

8. The method of claim 7, further comprising administering an antigen or vaccine to the subject.

9. A method of treating or preventing a viral infection comprising administering an effective amount of a pharmaceutical composition of claim 5 to a subject in need thereof, optionally in combination with a vaccine.

10. A method of treating or preventing cancer comprising administering autologous blood cells activated with a conjugate of claim 1 to a subject in need thereof.

11. A product produced by mixing peripheral blood cells with a conjugate of claim 1.

12. A method of treating or preventing cancer comprising administering an effective amount of a product of claim 11 to subject.

13. A nucleic acid encoding a GM-CSF and IL-9 conjugate.

14. A recombinant vector comprising a nucleic acid of claim 13.

15. An expression system configured to produce GM-CSF and IL-9 conjugate comprising the recombinant vector of claim 14.


SEQUENCE LISTING (3 sequences)

SEQ ID NO: 1 - protein, 288 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEMLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 2 - protein, 285 aa, Homo sapiens
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKMLVTYILASVLLFSSVLGQRCSTTWGIRDTNYLIENLKDDPPSKCSCSGNVTSCLCLSVPTDDCTTPCYREGLLQLTNATQKSRLLPVFHRVKRIVEVLKNITCPSFSCEKPCNQTMAGNTLSFLKSLLGTFQKTEMQRQKSRP

SEQ ID NO: 3 - protein, 867 aa, Homo sapiens
ATGTGGCTGCAGAGCCTGCTGCTCTTGGGCACTGTGGCCTGCAGCATCTCTGCACCCGCCCGCTCGCCCAGCCCCAGCACGCAGCCCTGGGAGCATGTGAATGCCATCCAGGAGGCCCGGCGTCTCCTGAACCTGAGTAGAGACACTGCTGCTGAGATGAATGAAACAGTAGAAGTCATCTCAGAAATGTTTGACCTCCAGGAGCCGACCTGCCTACAGACCCGCCTGGAGCTGTACAAGCAGGGCCTGCGGGGCAGCCTCACCAAGCTCAAGGGCCCCTTGACCATGATGGCCAGCCACTACAAGCAGCACTGCCCTCCAACCCCGGAAACTTCCTGTGCAACCCAGATTATCACCTTTGAAAGTTTCAAAGAGAACCTGAAGGACTTTCTGCTTGTCATCCCCTTTGACTGCTGGGAGCCAGTCCAGGAGATGCTTCTGGCCATGGTCCTTACCTCTGCCCTGCTCCTGTGCTCCGTGGCAGGCCAGGGGTGTCCAACCTTGGCGGGGATCCTGGACATCAACTTCCTCATCAACAAGATGCAGGAAGATCCAGCTTCCAAGTGCCACTGCAGTGCTAATGTGACCAGTTGTCTCTGTTTGGGCATTCCCTCTGACAACTGCACCAGACCATGCTTCAGTGAGAGACTGTCTCAGATGACCAATACCACCATGCAAACAAGATACCCACTGATTTTCAGTCGGGTGAAAAAATCAGTTGAAGTACTAAAGAACAACAAGTGTCCATATTTTTCCTGTGAACAGCCATGCAACCAAACCACGGCAGGCAACGCGCTGACATTTCTGAAGAGTCTTCTGGAAATTTTCCAGAAAGAAAAGATGAGAGGGATGAGAGGCAAGATATGA